(19) United States
(12) Patent Application Publication
(10) Pub. No.: US 2015/0314007 A1
(43) Pub. Date: Nov. 5, 2015

(54) LINKER-PAYLOAD MOLECULE CONJUGATES

(71) Applicant: GLYKOS FINLAND OY, Helsinki (FI)

(72) Inventors: Tero SATOMAA, Helsinki (FI); Jari HELIN, Rajamäki (FI); Filip S. EKHOLM, Porvoo (FI); Henna PYNNÖNEN, Vantaa (FI)

(73) Assignee: Glykos Finland Oy, Helsinki (FI)

(21) Appl. No.: 14/654,034

(22) PCT Filed: Dec. 20, 2013
(86) PCT No.: PCT/FI2013/051193

§ 371 Date: Jun. 19, 2015

Related U.S. Application Data
(60) Provisional application No. 61/740,662, filed on Dec. 21, 2012.
(60) Provisional application No. 61/740,686, filed on Dec. 21, 2012.

(30) Foreign Application Priority Data
Dec. 21, 2012 (FI) 20126364
Oct. 14, 2013 (FI) 20136019

(51) Int. Cl. A61K 47/48; A61K 38/08; C07K 7/06; C07K 9/00; C07K 16/28

(52) U.S. Cl. CPC A61K 47/48092; C07K 9/001; C07K 16/2863; C07K 7/06; A61K 38/08; A61K 47/48438; A61K 47/48561; A61K 47/48338; C07K 2317/24; C07K 2317/73

(57) ABSTRACT

The invention relates to novel linker-payload molecule conjugates. The invention also relates to novel cell binder-linker-payload molecule conjugates, in particular antibody conjugates of dolastatin or auristatin derivatives.

### FIELD OF THE INVENTION

The invention relates to novel linker-payload molecule conjugates. The invention also relates to novel cell binder-linker-payload molecule conjugates.

### BACKGROUND OF THE INVENTION

Conjugates of payload molecules such as cytotoxic drugs with linkers, used e.g. in making cell binder-linker-payload molecule conjugates that are useful, for instance, in therapy of cancer, have been described e.g. in Dosio et al., Toxins 2011, 3, 848-883, and Sammet et al., Pharm. Pat. Analyst 2012, 1(1), 2046-8954. The linkers described therein utilize various chemistries; however, many of them may not be optimal in terms of e.g. activity of the payload molecule, aqueous solubility of the conjugate or the reaction conditions required for conjugation.

### PURPOSE OF THE INVENTION

The purpose of the present invention is to provide linker-payload molecule conjugates and cell binder-linker-payload molecule conjugates that have improved properties as compared to known conjugates and that retain high activity of the payload molecule.

### SUMMARY

The linker-payload molecule conjugate according to the present invention is characterized by what is presented in claim 1, 5 or 6.

The cell binder-linker-payload molecule conjugate according to the present invention is characterized by what is presented in claim 4.

The pharmaceutical composition according to the present invention is characterized by what is presented in claim 33.

The method for modulating growth of a cell population according to the present invention is characterized by what is presented in claim 34.

The method for preparing a linker-payload molecule conjugate according to the present invention is characterized by what is presented in claim 35.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 shows the in vitro cytotoxicity of dolastatin derivatives against ovarian cancer cell line SKOV-3 as viability % compared to control cells (y-axis) measured at different derivative concentrations in the medium (x-axis);

FIG. 2 shows in vitro cytotoxicity assays of antibody-drug conjugates and drug derivatives;

FIG. 3 demonstrates tumor volume in subcutaneous SKOV-3 xenograft mice treated with anti-HER2 antibody-drug conjugate (ADC; drug-to-antibody ratio DAR=1 or DAR=3), anti-HER2 antibody or PBS (control); and

FIG. 4 shows tumor volume in subcutaneous SKOV-3 xenograft mice treated with anti-EGFR1 ADC, anti-EGFR1 IgG antibody or PBS (control).

### DETAILED DESCRIPTION OF THE INVENTION

The present invention provides linkers that are conjugated to a suitable group such as an amine group in a payload molecule by alkylation. In one embodiment, the present invention provides hydrophilic linkers comprising one or more hydroxyl groups that are conjugated to a suitable group such as an amine group in a payload molecule by alkylation.

The conjugation of the linker by alkylation to form a secondary or tertiary amine in the payload molecule provides several advantages, such as i) retaining of the chemical characteristics of the amine group such as basicity, in contrast to e.g. amide bond formation, ii) formation of a flexible and relatively small chemical group in contrast to e.g. rigid and bulky amide bond, iii) high specificity of the alkylation reaction, and iv) relatively mild reaction conditions suitable for labile payload molecules.

The presence of one or more hydroxyl groups in the hydrophilic linker provides several advantages, such as i) higher water solubility of the final product, ii) ability to use higher concentrations of the conjugate in aqueous solutions, iii) ability to link a higher number of payload molecules per molecule of cell binder, iv) ability to achieve higher hydrophilic conjugate concentration inside the target cell, and v) improved sensitivity of multidrug resistant cells.

In this context, the term “linker” and “linker according to the invention” should be understood as referring to the moiety or portion of a molecule represented by any one of formulas I, II, III, IV or V that does not comprise the payload molecule D; or wherein a molecule is represented by formula IV, the moiety or portion of the molecule that does not comprise the payload molecule D and the cell binder B.

The present invention relates to a linker-payload molecule conjugate represented by formula I:

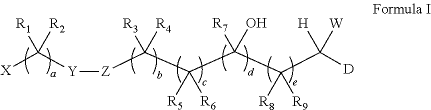

wherein

X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;

Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;

Z is a saccharide or absent;

D is a payload molecule comprising an amine moiety, through which the payload molecule is bound so as to form a secondary or tertiary amine;

R₁, R₂, R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;

W is H, CH₂OH, CH₃, carboxyl, substituted carboxyl, C₁-C₆ alkyl or substituted C₁-C₆ alkyl;

a is an integer from 0 to 6;

b is 0 or 1;

c and e are each independently an integer from 0 to 7; and

d is an integer from 1 to 7.

In one embodiment, the linker-payload molecule conjugate is represented by formula II

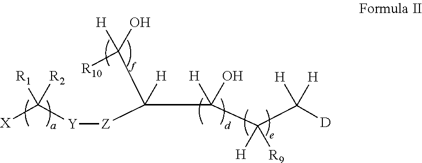

wherein

X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;

Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;

Z is a saccharide or absent;

D is a payload molecule comprising an amine moiety, through which the payload molecule is bound so as to form a secondary or tertiary amine;

R₁, R₂, R₉ and R₁₀ are each independently H, hydroxyl, amine, C₂-C₆ acylamide, carboxyl, substituted carboxyl, C₁-C₆ alkyl or substituted C₁-C₆ alkyl;

a is an integer from 0 to 6;

e is an integer from 0 to 3; and

d and f are integers from 0 to 4 with the proviso that their sum is from 1 to 4.

In one embodiment, the linker-payload molecule conjugate is represented by formula III

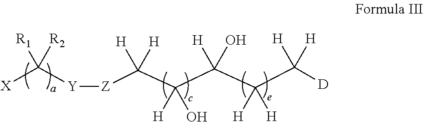

wherein

X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;

Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;

Z is a saccharide or absent;

D is a payload molecule comprising an amine moiety, through which the payload molecule is bound so as to form a secondary or tertiary amine;

R₁ and R₂ are each independently H, hydroxyl, amine, C₂-C₆ acylamide, carboxyl, substituted carboxyl, C₁-C₆ alkyl or substituted C₁-C₆ alkyl;

a is an integer from 0 to 6; and

c and e are each independently an integer from 0 to 3.

The present invention also relates to a cell binder-linker-payload molecule conjugate represented by formula IV

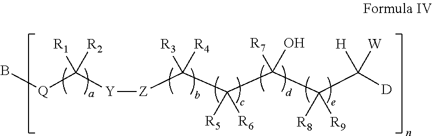

wherein

Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;

Z is a saccharide or absent;

D is a payload molecule comprising an amine moiety, through which the payload molecule is bound so as to form a secondary or tertiary amine;

R₁, R₂, R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;

W is H, CH₂OH, CH₃, carboxyl, substituted carboxyl, C₁-C₆ alkyl or substituted C₁-C₆ alkyl;

a is an integer from 0 to 6;

b is 0 or 1;

c and e are each independently an integer from 0 to 7; and

d is an integer from 1 to 7

B is a cell binder;

Q is E′-F′-E, wherein F′ is an amine, amide, disulfide, thioether, thioester, hydrazone, Schiff base, oxime, olefin metathesis reaction product, triazole or phosphine group, or other group generated by the reaction of the cell binder with F as defined for formula I, and E and E′ are each independently either absent or a polyethyleneoxy unit of formula (CH₂CH₂O)ₚ, wherein p is an integer from 2 to about 20; and

n is an integer from 1 to about 20.

A skilled person will recognise that the linker-payload molecule conjugate moiety linked to a cell binder as represented in formula IV is essentially the same as represented by formula I. In the cell binder-linker-payload molecule conjugate, the cell binder, B, and the payload, D, have thus reacted at the two ends of the linker. Using the linkers according to the invention, one or more amine-conjugated payload molecules can be introduced to a cell binder. Using the hydrophilic linkers according to the invention comprising hydroxyl group(s), a higher number of payload molecules can be introduced.

In this context, the term “cell binder” should be understood as referring to an agent capable of recognising and binding a target cell, for instance a cancer cell. Examples of known cell binders the use of which in cell binder-linker-payload conjugates have previously been described in e.g. Sammet et al., Pharm. Pat. Analyst 2012, 1(1), 65-73.

In one embodiment of the present invention, the cell binder comprises at least one amine, thiol, azide, alkene, alkyne, aldehyde, ketone, carboxylic acid or hydroxylamine group through which it is bound.

In one embodiment of the present invention, the cell binder comprises a molecule selected from the group consisting of a peptide, saccharide, small molecule and nucleic acid.

In one embodiment of the present invention, the linker-payload molecule conjugate is bound to an α-amino group or ε-amino group of a lysine in the peptide.

In one embodiment of the present invention, the peptide is a protein such as an antibody, lectin, growth factor, transferrin, or the like, that is capable of interacting with an internalization receptor on a cell surface.

In one embodiment of the present invention, the cell binder comprises an antibody or a fragment thereof.

In one embodiment of the present invention, the cell binder is an antibody or a fragment thereof.

In one embodiment of the present invention, the cell binder comprises a monoclonal or recombinant antibody or a fragment thereof.

In one embodiment of the present invention, the cell binder is a monoclonal or recombinant antibody or a fragment thereof.

In one embodiment of the present invention, the antibody or a fragment thereof is directed against human vascular endothelial growth factor (VEGF), epidermal growth factor receptor 1 (EGFR), tumor necrosis factor alpha (TNF-α), CD20, epidermal growth factor receptor 2 (HER2/neu), CD52, CD33, CD11a, glycoprotein IIb/IIIa, CD25, IgE, IL-2 receptor, or respiratory syncytial virus (RSV).

In one embodiment of the present invention, the antibody a fragment thereof is directed against human vascular endothelial growth factor (VEGF), epidermal growth factor receptor 1 (EGFR), tumor necrosis factor alpha (TNF-α), CD20, CD22, HIV-1 envelope glycoprotein gp120, cancer-associated high-mannose type N-glycans, epidermal growth factor receptor 2 (HER2/neu), CD52, CD33, CD11a, glycoprotein IIb/IIIa, CD25, IgE, IL-2 receptor, or respiratory syncytial virus (RSV).

In other embodiments, the antibody or a fragment thereof is directed against D19, CD21, CD22, CD30, CD38, CD40, CD70, CD74, CD83, CD133, CD138, CD200, or CD276. In other embodiments, the antibody or a fragment thereof is directed against 2G12 (HIV-1 envelope glycoprotein gp120), cancer-associated high-mannose type N-glycans, BMPR1B, LAT1 (SLC7A5), STEAP1, MUC16, MUC1, megakaryocyte potentiating factor (MPF), Napi3b, Sema 5b, PSCA hlg, ETBR (Endothelin type B receptor), STEAP2, TrpM4, CRIPTO, CD21, CD79a, CD79b, FcRH2, HER3, HER4, NCA, MDP, IL20Rα, brevi-can, Ephb2R, ASLG659, PSCA, PSMA, TMPRSS2, TMPRSS4, GEDA, BAFF-R, CXCRS, HLA-DOB, P2X5, CD72, LY64, FCRH1, PLAC1, VEGFR1, VEGFR2, or IRTA2. In other embodiments, the antibody or a fragment thereof is directed against CD2, CD3, CD3E, CD4, CD11, CD14, CD16, CD18, CD19, CD23, CD28, CD29, CD30, CD32, CD40L, CD51, CD54, CD56, CD70, CD80, CD123, CD133, CD138, CD147, CD227, or CD276. In other embodiments, the antibody or a fragment thereof directed against IL-1, IL1R, IL-2, IL-2R, IL-4, IL-5, IL-6, IL-6R, IL-8, IL-12, IL-15, IL-18, or IL-23. In other embodiments, the antibody or a fragment thereof is directed against a protein from the solute carrier family of proteins (e.g., solute carrier family 44, member 4 (protein encoded by SLC44A4 gene) or solute carrier family 34, member 2 (protein encoded by the SLC34A2 gene)); LIV-1 (protein encoded by SLC39A6 gene); protein from the SLAM family of proteins (e.g., SLAM family members 1, 2, 3, 4, 5, 6, 7, 8 or 9); protein from the mucin family of proteins (e.g., MUC1, MUC2, MUC3, MUC4, MUC5, MUC6, MUC7, MUCK, MUC9, MUC10, MUC11, MUC12, MUC13, MUC14, MUC15, or MUC16); protein from the STEAP family of proteins (e.g., STEAP1, STEAP2, STEAP3 or STEAP4); a protein from the tumor necrosis factor receptor family (e.g., TNF-RI, TNF-RII, DR1, DR2, DR3, DR4, DR5); glycoprotein IIb/IIIa; MN protein; mesothelin protein; protein encoded by the Slitrk family of proteins (e.g., SLITRK1, SLITRK2, SLITRK3, SLITRK4, SLITRK5, or SLITRK6), or a protein encoded by the GPNMB gene. In other embodiments, the antibody or a fragment thereof is directed against alpha-fetoprotein, angiopoietin 2, CA-125, carbonic anhydrase 9, CCR4, CD140a, CD152, CD174 (Lewis y), CD221, CD44, CD44v6, CEA, CTLA-4, DLL4, EGFL7, EpCAM, fibronectin extra domain-B, folate receptor 1, Frizzled receptor, GD2, GD3, glycoprotein 75, HGF, HLA-DR, human scatter factor receptor kinase, IGF-1 receptor, IGF-1, IgG4, ILGF2, ILGF1R, integrin α5β1, integrin αvβ3, MCP-1, MS4A1, Neu5Gc, PD-1, PDGF-R, phosphatidylserine, RON, SDC1, TAG-72, tenascin C, TRAIL-R1, TRAIL-R2, TWEAK receptor, or vimentin. However, these antibody targets are provided as examples only, to which the invention is not limited; a skilled person will appreciate that the antibody or a fragment thereof of the invention is not limited to any particular antibody or form thereof.

In one embodiment, the target molecule is EGFR. In other words, the antibody-drug conjugate is an anti-EGFR conjugate.

In one embodiment, the target molecule is epidermal growth factor receptor 1 (EGFR) having a sequence set forth in SEQ ID NO: 1. In one embodiment, anti-EGFR antibody is cetuximab.

Neoplastic diseases or cancers for the treatment of which the anti-EGFR antibody drug conjugates of the invention can be employed are EGFR-overexpressing tumours, respiratory tract tumours (e.g. parvicellular and non-parvicellular carcinomas, bronchial carcinoma), including preferably non-parvicellular carcinoma of the lung; tumours of the digestive organs (e.g. oesophagus, stomach, gall bladder, small intestine, large intestine, rectum), including especially intestinal tumours; tumours of the endocrine and exocrine glands (e.g. thyroid and parathyroid glands, pancreas and salivary gland), including preferably pancreas; tumours of the head and neck region (e.g. larynx, hypopharynx, nasopharynx, oropharynx, lips, oral cavity, tongue and oesophagus); and/or gliomas.

In one embodiment, the target molecule is HER2 having a sequence set forth in SEQ ID NO: 2.

In one embodiment of the present invention, the antibody is bevacizumab, tositumomab, etanercept, trastuzumab, adalimumab, alemtuzumab, gemtuzumab ozogamicin, efalizumab, rituximab, infliximab, abciximab, basiliximab, palivizumab, omalizumab, daclizumab, cetuximab, panitumumab, ibritumomab tiuxetan, abagovomab, actoxumab, adecatumumab, afutuzumab, altumomab, amatuximab, anifrolumab, apolizumab, atinumab, atlizumab, atorolimumab, bapineuzumab, basiliximab, bavituximab, belimumab, benralizumab, bertilimumab, besilesomab, bezlotoxumab, bimagrumab, bivatuzumab, blinatumomab, blosozumab, brentuximab, briakinumab, brodalumab, canakinumab, cantuzumab, caplacizumab, capromab, carlumab, catumaxomab, CC49, cedelizumab, cixutumumab, clazakizumab, clenoliximab, clivatuzumab, conatumumab, concizumab, crenezumab, CR6261, dacetuzumab, dalotuzumab, daratumumab, demcizumab, denosumab, detumomab, drozitumab, duligotumab, dupilumab, dusigitumab, ecromeximab, eculizumab, edobacomab, edrecolomab, eldelumab, elotuzumab, elsilimomab, enavatuzumab, enlimomab, enokizumab, enoticumab, ensituximab, epitumomab, epratuzumab, ertumaxomab, etaracizumab, etrolizumab, evolocumab, exbivirumab, fanolesomab, faralimomab, farletuzumab, fasinumab, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gevokizumab, girentuximab, glembatumumab, golimumab, gomiliximab, guselkumab, ibalizumab, icrucumab, imciromab, imgatuzumab, inclacumab, indatuximab, intetumumab, inolimomab, inotuzumab, ipilimumab, iratumumab, itolizumab, ixekizumab, keliximab, labetuzumab, lambrolizumab, lampalizumab, lebrikizumab, lemalesomab, lerdelimumab, lexatumumab, libivirumab, ligelizumab, lintuzumab, lirilumab, lodelcizumab, lorvotuzumab, lucatumumab, lumiliximab, mapatumumab, margetuximab, maslimomab, mavrilimumab, matuzumab, mepolizumab, metelimumab, milatuzumab, minretumomab, mitumomab, mogamulizumab, morolimumab, motavizumab, moxetumomab, muromonab, namilumab, narnatumab, natalizumab, nebacumab, necitumumab, nerelimomab, nesvacumab, nimotuzumab, nivolumab, obinutuzumab, ocaratuzumab, ocrelizumab, odulimomab, ofatumumab, olaratumab, olokizumab, onartuzumab, oregovomab, orticumab, otelixizumab, oxelumab, ozanezumab, ozoralizumab, pagibaximab, panobacumab, parsatuzumab, pascolizumab, pateclizumab, patritumab, pemtumomab, perakizumab, pertuzumab, pidilizumab, pinatuzumab, pintumomab, placulumab, polatuzumab, ponezumab, priliximab, pritoxaximab, pritumumab, quilizumab, racotumomab, radretumab, rafivirumab, ramucirumab, raxibacumab, regavirumab, reslizumab, rilotumumab, robatumumab, roledumab, romosozumab, rontalizumab, rovelizumab, ruplizumab, samalizumab, sarilumab, satumomab, secukinumab, seribantumab, setoxaximab, sevirumab, sibrotuzumab, sifalimumab, siltuximab, simtuzumab, siplizumab, sirukumab, solanezumab, solitomab, sonepcizumab, sontuzumab, stamulumab, suvizumab, tabalumab, tacatuzumab, talizumab, tanezumab, taplitumomab, tefibazumab, tenatumomab, teneliximab, teplizumab, teprotumumab, TGN1412, ticilimumab, tildrakizumab, tigatuzumab, tocilizumab, toralizumab, tovetumab, tralokinumab, TRBS07, tregalizumab, tremelimumab, tucotuzumab, tuvirumab, ublituximab, urelumab, urtoxazumab, ustekinumab, vantictumab, vapaliximab, vatelizumab, vedolizumab, veltuzumab, vepalimomab, vesencumab, visilizumab, volociximab, vorsetuzumab, votumumab, zalutumumab, zanolimumab, zatuximab, ziralimumab, or zolimomab. However, these antibodies are provided as examples only, to which the invention is not limited; a skilled person will appreciate that the antibody of the invention is not limited to any particular antibody or form thereof.

In one embodiment of the present invention, the cell binder is the antibody bevacizumab (available e.g. under the trademark AVASTIN®), tositumomab (BEXXAR®), etanercept (ENBREL®), trastuzumab (HERCEPTIN®), adalimumab (HUMIRA®), alemtuzumab (CAMPATH®), gemtuzumab ozogamicin (MYLOTARG®), efalizumab (RAPTIVE®), rituximab (RITUXAN®), infliximab (REMICADE®), abciximab (REOPRO®), basiliximab (SIMULECT®), palivizumab (SYNAGIS®), omalizumab (XOLAIR®), daclizumab (ZENAPAX®), cetuximab (ERBITUX®), panitumumab (VECTIBIX®) or ibritumomab tiuxetan (ZEVALIN®).

In one embodiment of the present invention, the antibody is cetuximab, trastuzumab, panitumumab, rituximab, bevacizumab, tositumomab, etanercept, adalimumab, alemtuzumab, gemtuzumab ozogamicin, efalizumab, rituximab, infliximab, abciximab, basiliximab, palivizumab, omalizumab, daclizumab, epratuzumab, lintuzumab, nimotuzumab or ibritumomab tiuxetan.

In one embodiment, the antibody is cetuximab. In one embodiment, cetuximab has a sequence set forth in SEQ ID NO:s 3 and 4.

In one embodiment, the antibody is trastuzumab. In one embodiment, trastuzumab has a sequence set forth in SEQ ID NO:s 5 and 6.

In one embodiment, the antibody is rituximab. In one embodiment, rituximab has a sequence set forth in SEQ ID NO:s 7 and 8.

In one embodiment, the antibody is bevacizumab. In one embodiment, bevacizumab has a sequence set forth in SEQ ID NO:s 9 and 10. In one embodiment, the antibody is etanercept.

In one embodiment, etanercept has a sequence set forth in SEQ ID NO: 11.

In one embodiment, the antibody is adalimumab. In one embodiment, adalimumab has a sequence set forth in SEQ ID NO:s 12 and 13.

In one embodiment, the antibody is alemtuzumab. In one embodiment, alemtuzumab has a sequence set forth in SEQ ID NO:s 14 and 15.

In one embodiment, the antibody is efalizumumab. In one embodiment, efalizumab has a sequence set forth in SEQ ID NO:s 16 and 17.

In one embodiment, the antibody is infliximab. In one embodiment, infliximab has a sequence set forth in SEQ ID NO:s 18 and 19.

In one embodiment, the antibody is basiliximab. In one embodiment, basiliximab has a sequence set forth in SEQ ID NO:s 20 and 21.

In one embodiment, the antibody is omalizumab. In one embodiment, omalizumab has a sequence set forth in SEQ ID NO:s 22 and 23.

In one embodiment, the antibody is daclizumab. In one embodiment, daclizumab has a sequence set forth in SEQ ID NO:s 24 and 25.

In one embodiment, the antibody is nimotuzumab. In one embodiment, nimotuzumab has a sequence set forth in SEQ ID NO:s 26 and 27.

In one embodiment, the antibody is epratuzumab. In one embodiment, epratuzumab has a sequence set forth in SEQ ID NO:s 28 and 29.

In one embodiment, the antibody is lintuzumab. In one embodiment, lintuzumab has a sequence set forth in SEQ ID NO:s 30 and 31.

In one embodiment, the antibody is 2G12. In one embodiment, 2G12 has a sequence set forth in SEQ ID NO:s 32 and 33.

In one embodiment, the antibody is ibritumomab tiuxetan.

In one embodiment, the antibody is tositumomab.

In one embodiment, the antibody is panitumumab.

In one embodiment, the antibody is gemtuzumab ozogamicin.

In one embodiment, the antibody is palivizumab.

In one embodiment, the antibody is abciximab.

In one embodiment of the present invention, the cell binder is a fusion protein comprising an Fc domain, or a fragment thereof. Said fusion protein may, in addition to the Fc domain, or a fragment thereof, comprise e.g. a receptor moiety having a different biological function. Fusion protein should also be understood as meaning antibody like molecules which combine the “binding domain” of a heterologous “adhesin” protein (e.g. a receptor, ligand or enzyme) with an Fc domain. Structurally, these immunoadhesins comprise a fusion of the adhesin amino acid sequence with the desired binding specificity which is other than the antigen recognition and binding site (antigen combining site) of an antibody (i.e. is “heterologous”) and an Fc domain sequence. Examples of immunoadhesins include but are not limited to etanercept (available e.g. under the trade mark ENBREL®), which is a soluble TNF receptor 2 protein fused to the Fc region of human IgG1, carcio-nembryonic antigen-immunoglobulin Fc fusion protein (CEA-Fc) and factor IX-Fc fusion protein.

In one embodiment of the present invention, the peptide is capable of binding to an internalizing receptor on a cell surface either directly or indirectly. In one embodiment, the peptide comprises the thrombospondin 1-binding tetrapeptide Leu-Ser-Lys-Leu. In one embodiment, the peptide is the thrombospondin 1-binding tetrapeptide Leu-Ser-Lys-Leu.

In one embodiment of the present invention, the saccharide is capable of binding to an internalizing receptor on a cell surface either directly or indirectly. In some embodiments, the saccharide comprises non-reducing terminal β-galactose, Galβ1-4GlcNAc or α-Neu5Ac; capable of binding to e.g. galectins, asialoglycoprotein receptor or siglecs, respectively. A person skilled in the art will understand that the number of payload molecules bound to a single cell binder will vary depending on e.g. the cell binder and conditions of the conjugation reaction. In one embodiment of the present invention, n, i.e. the number of payload molecules bound to a single cell binder, is 1-20. In one embodiment of the present invention, n is 2-18. In one embodiment of the present invention, n is 2-16. In one embodiment of the present invention, n is 2-10. In other embodiments, n is 2-6; 2-5; 2-4; 2-3; 3-4; or 1, 2, 3 or 4.

In one embodiment of the present invention, the cell binder-linker-payload molecule conjugate is selected from the group consisting of compounds of the following formulas:

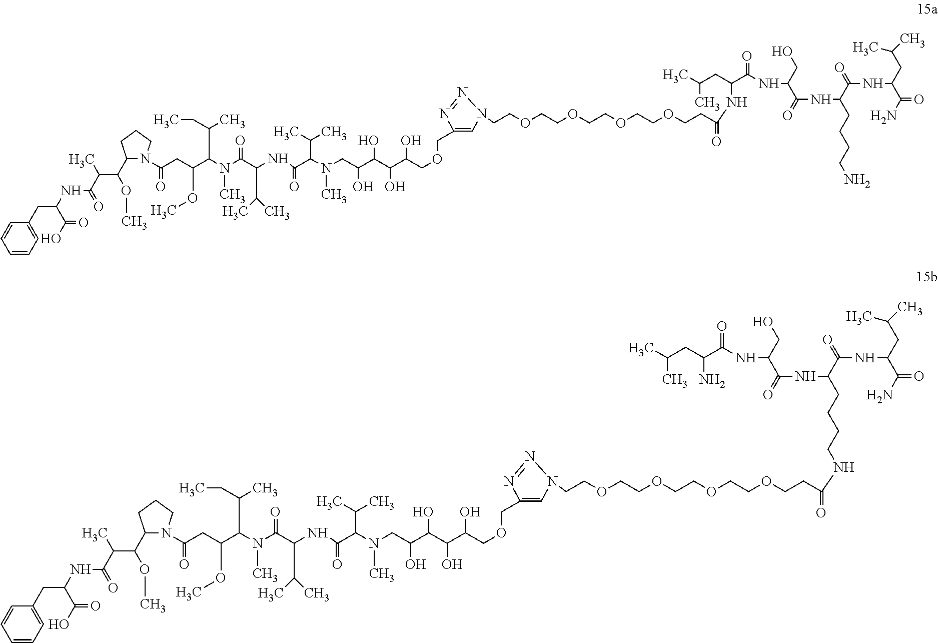 [SMILES: CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NC(CC(C)C)C(=O)NC(CO)C(=O)NC(CCCCN)C(=O)NC(CC(C)C)C(N)=O)nn1)C(C)C ; CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NCCCCC(NC(=O)C(CO)NC(=O)C(N)CC(C)C)C(=O)NC(CC(C)C)C(N)=O)nn1)C(C)C]

In one embodiment of the present invention, the cell binder-linker-payload molecule conjugate is a conjugate generated by azide-alkyne cycloaddition reaction between cetuximab-PEG₄-N₃ and N-(6-propargyl-D-galactosyl)-dolastatin 10. The generation of said conjugate is described in Example 6.

In one embodiment of the present invention, the cell binder-linker-payload molecule conjugate is a conjugate generated by azide-alkyne cycloaddition reaction between omalizumab-PEG₄-N₃ and N-(6-propargyl-D-galactosyl)-dolastatin 10. The generation of said conjugate is described in Example 6.

In one embodiment of the present invention, the cell binder-linker-payload molecule conjugate is a conjugate generated by azide-alkyne cycloaddition reaction between 2G12-PEG₄-N₃ and N-(6-propargyl-D-galactosyl)-dolastatin 10. The generation of said conjugate is described in Example 7.

In one embodiment of the present invention, the cell binder-linker-payload molecule conjugate is a conjugate generated by azide-alkyne cycloaddition reaction between trastuzumab-PEG₄-N₃ and N-(6-propargyl-D-galactosyl)-dolastatin 10. The generation of said conjugate is described in Example 8.

The present invention also relates to a linker-payload molecule conjugate represented by formula V

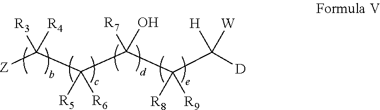

wherein Z is H, OH or a saccharide;

D is a payload molecule comprising an amine moiety, through which the payload molecule is bound so as to form a secondary or tertiary amine;

R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;

W is H, CH₂OH, CH₃, carboxyl, substituted carboxyl, C₁-C₆ alkyl or substituted C₁-C₆ alkyl;

b is 0 or 1;

c and e are each independently an integer from 0 to 7; and

d is an integer from 1 to 7.

In one embodiment of the present invention, the saccharide comprises a glycosidic bond that is cleavable by a lysosomal glycosidase. In some embodiments, the glycosidase is a lysosomal β-galactosidase, β-hexosaminidase, β-glucuronidase, α-galactosidase, α-glucosidase, α-mannosidase, β-mannosidase, α-fucosidase or neuraminidase; and the glycosidic bond is the anomeric bond of a pyranose monosaccharide comprised in the saccharide that is or is analogous to β-D-galactose, N-acetyl-β-D-galactosamine, N-acetyl-β-D-glucosamine, β-D-glucuronic acid, α-D-galactose, α-D-glucose, α-D-mannose, β-D-mannose, α-L-fucose or neuraminic acid, respectively. This embodiment has the added utility that a lysosomal glycosidase may release the payload molecule in active form inside a cell. In some embodiments of the present invention, the released payload molecule may be more potent and/or active inside a cell.

In this context, the term “being analogous to” means that the analogous pyranose monosaccharide is cleavable by the same glycosidase than the other pyranose monosaccharide to which it is analogous to.

In one embodiment of the present invention, the activity of the lysosomal glycosidase releases the payload molecule in active form inside a cell.

In one embodiment of the present invention, one or more of the glycosidic bonds of the saccharide are essentially stable in neutral pH and/or in serum.

In one embodiment of the present invention, all glycosidic bonds of the saccharide are essentially stable in neutral pH and/or in serum.

The present invention also relates to a linker-payload molecule conjugate represented by formula VI

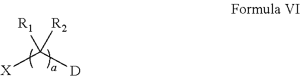

wherein

X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;

D is a payload molecule comprising an amine moiety, through which the payload molecule is bound so as to form a secondary or tertiary amine,

wherein the payload molecule is a dolastatin 10 or a derivative thereof, or dolastatin 15 or a derivative thereof;

R₁ and R₂ are each independently H, hydroxyl, amine, C₂-C₆ acylamide, carboxyl, substituted carboxyl, C₁-C₆ alkyl or substituted C₁-C₆ alkyl; and

a is an integer from 1 to 20.

In one embodiment of the present invention, D is a payload molecule comprising an amine moiety, through which the payload molecule binds to the carbon atom having the substituent W so as to form a secondary or tertiary amine.

In one embodiment of the invention, X is an amine reacting group, a thiol reactive group, an azide reactive group, an alkyne reactive group, a carbonyl reactive group or a hydroxylamine reactive group.

In one embodiment of the invention, X is an amine reacting group, such as (but not limited) to an N-hydroxysuccinmide ester, p-nitrophenyl ester, dinitrophenyl ester, or pentafluorophenyl ester.

In one embodiment of the invention, X is a thiol reactive group, such as (but not limited to) pyridyldisulfide, nitropyridyldisulfide, maleimide, haloacetate or carboxylic acid chloride.

In one embodiment of the invention, X is a azide reactive group, such as (but not limited to) alkyne.

In one embodiment of the present invention, X is an alkyne.

In one embodiment of the present invention, X is CH≡C.

In one embodiment of the invention, X is an alkyne reactive group, such as (but not limited to) azide.

In one embodiment of the present invention, X is azide.

In one embodiment of the invention, X is a carbonyl reactive group, such as (but not limited to) hydroxylamine.

In one embodiment of the invention, X is a hydroxylamine reactive group, such as (but not limited to) aldehyde or ketone.

In one embodiment of the invention, X is isothiocyanate, isocyanate, sulfonyl chloride, glyoxal, epoxide, oxirane, carbonate, aryl halide, imidoester, carbodiimide, or anhydride.

In one embodiment of the present invention, Z is absent.

In one embodiment of the present invention, Z is a saccharide.

In one embodiment of the present invention, Z is an oligosaccharide with a degree of polymerization from 1 to about 20; from 1 to 10; from 1 to 8; from 1 to 6; from 1 to 5; from 1 to 4; from 1 to 3; from 1 to 2; or 1, 2, 3, 4 or 5.

In one embodiment of the present invention, Z is a monosaccharide, disaccharide or trisaccharide.

In one embodiment of the present invention, Z is OH.

In one embodiment of the present invention, Z is H.

In one embodiment of the present invention, a is 1, 2, 3, 4, 5, or 6.

In one embodiment of the present invention, a is 1.

In one embodiment of the present invention, b is 0.

In one embodiment of the present invention, b is 1.

In one embodiment of the present invention, c is 0.

In one embodiment of the present invention, c is 1, 2, 3, 4, 5, 6 or 7.

In one embodiment of the present invention, d is 1, 2, 3, 4, 5, 6 or 7.

In one embodiment of the present invention, d is 3, 4 or 5.

In one embodiment of the present invention, d is 3.

In one embodiment of the present invention, d is 4.

In one embodiment of the present invention, d is 5.

In one embodiment of the present invention, d is 6.

In one embodiment of the present invention, e is 0.

In one embodiment of the present invention, e is 1, 2, 3, 4, 5, 6 or 7.

In one embodiment of the present invention, d is 3; and R₇ is H.

In one embodiment of the present invention, d is 4; and R₇ is H.

In one embodiment of the present invention, b is 1; and R₃ and R₄ are each H.

In one embodiment of the present invention, a is 1; and R₁ and R₂ are each H.

In one embodiment of the present invention, e is 1; and R₈ and R₉ are each H.

In one embodiment of the present invention, a, b, c, or e is 0.

In one embodiment of the present invention, a, b, c, and/or e is 0.

In one embodiment of the present invention, W is H.

In one embodiment of the present invention, a is 2 or 3; and R₁ and R₂ are both H.

In one embodiment of the present invention, Y is an oxygen.

In one embodiment of the present invention, Y is a sulphur.

In one embodiment of the present invention, Y is a peptide.

In one embodiment of the present invention, Y is a peptide that comprises an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide.

In one embodiment of the present invention, Y is a peptide that is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide.

In one embodiment of the present invention, Y is a peptide from 2 to 5 amino acids in length.

In one embodiment of the present invention, the peptide is linked to the linker through the terminal nitrogen i.e. through the amino terminus by an amide bond.

In one embodiment of the present invention, the peptide is linked to the linker through the terminal carbon i.e. through the carboxy terminus by an amide bond or an ester bond.

In one embodiment of the present invention, the peptide is linked to the linker through a side chain of one of the amino acids of the peptide by an amide, ester, disulfide or thioether bond.

In one embodiment of the present invention, the peptide comprises an amino acid sequence cleavable by a lysosomal peptidase, e.g. L-Gly-L-Gly, L-Val-L-Cit, L-Phe-L-Leu, L-Leu-L-Ala-L-Leu, L-Leu-L-Ala-L-Ala, L-Ala-L-Leu-L-Ala-L-Leu, and the like.

The term “alkyl” should be understood as referring to a straight or branched chain saturated or unsaturated hydrocarbon having the indicated number of carbon atoms (e.g., “C₁-C₈ alkyl” refers to an alkyl group having from 1 to 8 carbon atoms). When the number of carbon atoms is not indicated, the alkyl group has from 1 to 8 carbon atoms. Representative “C₁-C₈ alkyl” groups include (but are not limited to) methyl (Me, CH₃), ethyl (Et, CH₂CH₃), 1-propyl (n-Pr, n-propyl, CH₂CH₂CH₃), 2-propyl (i-Pr, isopropyl, CH(CH₃)₂), 1-butyl (n-Bu, n-butyl, CH₂CH₂CH₂CH₃), 2-methyl-1-propyl (i-Bu, isobutyl, CH₂CH(CH₃)₂), 2-butyl (s-Bu, s-butyl, CH(CH₃)CH₂CH₃), 2-methyl-2-propyl (t-Bu, tert-butyl, C(CH₃)₃), 1-pentyl (n-pentyl, CH₂CH₂CH₂CH₂CH₃), 2-pentyl (CH(CH₃)CH₂CH₂CH₃), 3-pentyl (CH(CH₂CH₃)₂), 2-methyl-2-butyl (C(CH₃)₂CH₂CH₃), 3-methyl-2-butyl (CH(CH₃)CH(CH₃) 2), 3-methyl-1-butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3-hexyl (CH(CH₂CH₃)(CH₂CH₂CH₃)), 2-methyl-2-pentyl (C(CH₃)₂CH₂CH₂CH₃), 3-methyl-2-pentyl (CH(CH₃)CH(CH₃)CH₂CH₃), 4-methyl-2-pentyl (CH(CH₃)CH₂CH(CH₃)₂), 3-methyl-3-pentyl (C(CH₃)(CH₂CH₃)₂), 2-methyl-3-pentyl (CH(CH₂CH₃)CH(CH₃)₂), 2,3-dimethyl-2-butyl (C(CH₃)₂CH(CH₃)₂), and 3,3-dimethyl-2-butyl (CH(CH₃)C(CH₃)₃). An alkyl group can be unsubstituted or substituted with one or more groups including, but not limited to, OH, O(C₁-C₈ alkyl), aryl, COR′, OCOR′, CONH₂, CONHR′, CONR′₂, NHCOR′, SH, SO₂R′, SOR′, OSO₂OH, OPO(OH)₂, halogen, N₃, NH₂, NHR′, NR′₂, NHCO(C₁-C₈ alkyl) or CN, wherein each R′ is independently either H, C₁-C₈ alkyl or aryl.

The term “alkyl” should also be understood as referring to an alkylene, a saturated, branched or straight chain or cyclic hydrocarbon radical of 1-18 carbon atoms, and having two monovalent radical centers derived by the removal of two hydrogen atoms from the same or two different carbon atoms of a parent alkane. Typical such alkylenes include (but are not limited to) methylene (CH₂) 1,2-ethyl (CH₂CH₂), 1,3-propyl (CH₂CH₂CH₂), 1,4-butyl (CH₂CH₂CH₂CH₂), and the like. The term “alkyl” should also be understood as referring to arylalkyl and heteroarylalkyl radicals as described below.

The term “alkenyl” should be understood as referring to a C₂-C₁₈ hydrocarbon containing normal, secondary, tertiary or cyclic carbon atoms with at least one site of unsaturation, i.e., a carbon-carbon, sp² double bond. Examples include, but are not limited to ethylene or vinyl (CH═CH₂), allyl (CH₂CH═CH₂), cyclopentenyl (C₅H₇), and 5-hexenyl (CH₂CH₂CH₂CH₂CH═CH₂). The term “alkenyl” should also be understood as referring to an alkenylene, an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to 1,2-ethylene (CH═CH).

The term “alkynyl” should be understood as referring to a C₂-C₁₈ hydrocarbon containing normal, secondary, tertiary or cyclic carbon atoms with at least one site of unsaturation, i.e., a carbon-carbon, sp triple bond. Examples include, but are not limited to acetylenic (C≡CH) and propargyl (CH₂C≡CH). The term “alkynyl” should also be understood as referring to an alkynylene, an unsaturated, branched or straight chain or cyclic hydrocarbon radical of 2-18 carbon atoms, and having two monovalent radical centers derived by the removal of two hydrogen atoms from carbon atoms of a parent alkyne. Typical alkynylene radicals include (but are not limited to) acetylene (C≡C), propargyl (CH₂C≡C), and 4-pentynyl (CH₂CH₂CH₂C≡C).

The term “aryl” should be understood as referring to a monovalent aromatic hydrocarbon radical of 6-20 carbon atoms derived by the removal of one hydrogen atom from a single carbon atom of a parent aromatic ring system. An aryl group can be unsubstituted or substituted. Typical aryl groups include (but are not limited to) radicals derived from benzene, substituted benzene, phenyl, naphthalene, anthracene, biphenyl, and the like. An aryl can be substituted with one or more groups including, but not limited to, OH, O(C₁-C₈ alkyl), aryl, COR′, OCOR′, CONH₂, CONHR′, CONR′₂, NHCOR′, SH, SO₂R′, SOR′, OSO₂OH, OPO(OH)₂, halogen, N₃, NH₂, NHR′, NR′₂, NHCO(C₁-C₈ alkyl) or CN, wherein each R′ is independently either H, C₁-C₈ alkyl or aryl. The term “aryl” should also be understood as referring to an arylene group which is an aryl group having two covalent bonds and can be in the para, meta, or ortho configurations, in which the phenyl group can be unsubstituted or substituted with up to four groups including but not limited to OH, O(C₁-C₈ alkyl), aryl, COR′, OCOR′, CONH₂, CONHR′, CONR′₂, NHCOR′, SH, SO₂R′, SOR′, OSO₂OH, OPO(OH)₂, halogen, N₃, NH₂, NHR′, NR′₂, NHCO(C₁-C₈ alkyl) or CN, wherein each R′ is independently either H, C₁-C₈ alkyl or aryl.

The term “arylalkyl” should be understood as referring to an acyclic alkyl radical in which one of the hydrogen atoms bonded to a carbon atom, typically a terminal or sp³ carbon atom, is replaced with an aryl radical. Typical arylalkyl groups include (but are not limited to) benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. The arylalkyl group comprises 6 to 20 carbon atoms, e.g., the alkyl moiety, including alkanyl, alkenyl or alkynyl groups, of the arylalkyl group is 1 to 6 carbon atoms and the aryl moiety is 5 to 14 carbon atoms.

The term “heteroarylalkyl” should be understood as referring to an acyclic alkyl radical in which one of the hydrogen atoms bonded to a carbon atom, typically a terminal or sp³ carbon atom, is replaced with a heteroaryl radical. Typical heteroarylalkyl groups include (but are not limited to) 2-benzimidazolylmethyl, 2-furylethyl, and the like. The heteroarylalkyl group comprises 6 to 20 carbon atoms, e.g., the alkyl moiety, including alkanyl, alkenyl or alkynyl groups, of the heteroarylalkyl group is 1 to 6 carbon atoms and the heteroaryl moiety is 5 to 14 ring atoms, typically 1 to 3 heteroatoms selected from N, O, P, and S, with the remainder being carbon atoms. The heteroaryl moiety of the heteroarylalkyl group may be a monocycle having 3 to 7 ring members (2 to 6 carbon atoms) or a bicycle having 7 to 10 ring members (4 to 9 carbon atoms) and 1 to 3 heteroatoms selected from N, O, P, and S, for example: a bicyclo[4,5], [5,5], [5,6], or [6,6] system.

The terms “substituted alkyl”, “substituted aryl” and “substituted arylalkyl” should be understood as referring to alkyl, aryl, and arylalkyl, respectively, in which one or more hydrogen atoms are each independently replaced with a substituent. Typical substituents include but are not limited to X, R, —O—, OR, SR, —S—, NR², NR³, ═NR, CX₃, CN, OCN, SCN, N═C═O, NCS, NO, NO₂, ═N₂, N₃, NRCOR, COR, CONR², —SO₃—, SO₃H, SO₂R, OSO₂OR, SO₂NR, SOR, OPO (OR)₂, PO(OR)₂, —PO₃—, PO₃H₂, COR, COX, C(═S)R, CO₂R, —CO₂—, C(═S)OR, COSR, C (═S)SR, CONR², C(═S)NR², and C(═NR)NR², where each X is independently a halogen: F, Cl, Br, or I; and each R is independently H, C₂-C₁₈ alkyl, C₆-C₂₀ aryl, C₃-C₁₄ heterocycle or protecting group. Alkylene, alkenylene, and alkynylene groups as described above may also be similarly substituted.

The terms “heteroaryl” and “heterocycle” should be understood as referring to a ring system in which one or more ring atoms is a heteroatom, e.g., nitrogen, oxygen, phosphate and sulfur. The heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960).

Examples of heterocycles include, by way of example and not limitation, pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl.

By way of example and not limitation, carbon-bonded heterocycles are bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl and 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole or isoindoline; position 4 of a morpholine; and position 9 of a carbazole or β-carboline. Still more typically, nitrogen bonded heterocycles include 1-aziridyl, 1-azetedyl, 1-pyrrolyl, 1-imidazolyl, 1-pyrazolyl and 1-piperidinyl.

The term “carbocycle” should be understood as referring to a saturated or unsaturated ring having 3 to 7 carbon atoms as a monocycle or 7 to 12 carbon atoms as a bicycle. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo [5,6] or [6,6] system. Examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cycloheptyl and cyclooctyl.

The term “saccharide” should be understood as referring to single simple sugar moieties or monosaccharides or their derivatives, as well as combinations of two or more single sugar moieties or monosaccharides covalently linked to form disaccharides, oligosaccharides, and polysaccharides. A saccharide can be a compound that includes one or more open chain or cyclized monomer units based upon an open chain form of compounds having the chemical structure H(CHOH)ₙC(═O) (CHOH)ₘH, wherein the sum of n+m is an integer in the range of 2 to 8. Thus, the monomer units can include trioses, tetroses, pentoses, hexoses, heptoses, octoses, nonoses, and mixtures thereof. One or several of the hydroxyl groups in the chemical structure can be replaced with other groups such as hydrogen, amino, amine, acylamido, acetylamido, halogen, mercapto, acyl, acetyl, phosphate or sulphate ester, and the like; and the saccharides can also comprise other functional groups such as carboxyl, carbonyl, hemiacetal, acetal and thio groups. Saccharides can include monosaccharides including, but not limited to, simple aldoses such as glyceraldehyde, erythrose, threose, ribose, arabinose, xylose, lyxose, allose, altrose, glucose, mannose, gulose, idose, galactose, talose and mannoheptulose; simple ketoses such as dihydroxyacetone, erythrulose, ribulose, xylulose, psicose, fructose, sorbose, tagatose and sedoheptulose; deoxysugars such as fucose, 2-deoxyglucose, 2-deoxyribose and rhamnose; sialic acids such as ketodeoxynonulosonic acid, N-acetylneuraminic acid and 9-O-acetyl-N-acetylneuraminic acid; uronic acids such as glucuronic acid, galacturonic acid and iduronic acid; amino sugars such as 2-amino-2-deoxygalactose and 2-amino-2-deoxyglucose; acylamino sugars such as 2-acetamido-2-deoxygalactose, 2-acetamido-2-deoxyglucose and N-glycolylneuraminic acid; phosphorylated and sulphated sugars such as 6-phosphomannose, 6-sulpho-N-acetylglucosamine and 3-sulphogalactose; and derivatives and modifications thereof. The term “saccharide” also includes non-reducing carbohydrates such as inositols and alditols and their derivatives. Saccharides according to the present invention may be in D- or L-configuration; in open-chain, pyranose or furanose form; α or β anomer; and any combination thereof.

Carbohydrate nomenclature in this context is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 293).

In this context, the terms “Neu5Ac”, “NeuNAc” and “neuraminic acid” refer to N-acetylneuraminic acid; “Gal” refers to D-galactose; “GlcNAc” refers to 2-acetamido-2-deoxy-D-glucose (N-acetyl-D-glucosamine); and all monosaccharide residues are in pyranose form and D-sugars except for L-fucose unless otherwise specified. In one embodiment of the present invention, “neuraminic acid” may also refer to other sialic acids in addition to N-acetylneuraminic acid, such as N-glycolylneuraminic acid (Neu5Gc).

The term “oligosaccharide” should be understood as referring to saccharides composed of two or several monosaccharides linked together by glycosidic bonds having a degree of polymerization in the range of from 2 to about 20. The term “oligosaccharide” should be understood as referring hetero- and homopolymers that can be either branched or linear and have a reducing end and a non-reducing end, whether or not the saccharide at the reducing end is in fact a reducing sugar. An oligosaccharide described herein may be described with the name or abbreviation for the non-reducing saccharide, followed by the configuration of the glycosidic bond (α or β), the ring bond, the ring position of the reducing saccharide involved in the bond, and then the name or abbreviation of the reducing saccharide, and so on (e.g. Galβ1-4Glc for lactose and Galα1-4Galβ1-4Glc for globotriose).

In one embodiment of the present invention, monosaccharides are in pyranose (P) or furanose (F) cyclized forms according to the formulas:

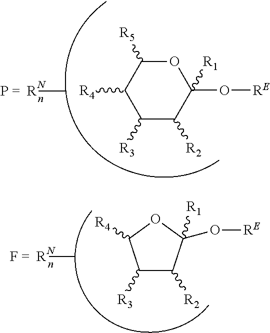

wherein R¹, R², R³, R⁴ and R⁵ groups are each independently either H, OH, CH₂OH, COOH, COOR′, C₁-C₈ alkyl, O(C₁-C₈ alkyl), aryl, COR′, OCOR′, CONH₂, CONHR′, CONR′₂, NHCOR′, SH, SO₂R′, SOR′, OSO₂OH, OPO (OH)₂, halogen, N₃, NH₂, NHR′, NR′₂, NHCO(C₁-C₈ alkyl) or R^{N}, wherein each R′ is independently either H, C₁-C₈ alkyl or aryl and each R^{N} is a non-reducing end saccharide; R^{E} is either H or reducing end structure such as a saccharide; n is an integer in the range of 0 to 3 in F or in the range of 0 to 4 in P; and the stereochemistry of each R¹, R², R³, R⁴ and R⁵ is dependent on the monosaccharide structure and its configuration and anomericity.

The term “disaccharide” should be understood as referring to a saccharide composed of two monosaccharides linked together by a glycosidic bond. Examples of disaccharides include, but are not limited to, lactose, N-acetyllactosamine, galactobiose, maltose, isomaltose and cellobiose.

The term “trisaccharide” should be understood as referring to a saccharide composed of three monosaccharides linked together by glycosidic bonds. Examples of trisaccharides include, but are not limited to, maltotriose, sialyllactose, globotriose, lacto-N-triose and gangliotriose.

The term “payload molecule” should be understood as referring to any molecule suitable for linkage using the linker according to the invention.

In one embodiment of the present invention, a payload molecule naturally comprises a primary or secondary amine moiety. In one embodiment of the present invention, a payload molecule is modified to comprise a primary or secondary amine moiety. In a preferred embodiment of the present invention, the amine-modified payload molecule essentially retains the activity of the original molecule.

In one embodiment of the present invention, D is a payload molecule which is a cytotoxic agent, or a labelling molecule, such as a fluorescent label or a radioactive label.

In one embodiment of the present invention, D is a cytotoxic agent.

In this context, the term “cytotoxic agent” should be understood as referring to a molecule that has the capability to affect the function or viability of a cell. The cytotoxic agent may be any compound that results in the death of a cell, or induces cell death, or in some manner decreases cell viability. The cytotoxic agent can be any of many small molecule drugs, including, but not limited to, dolastatins; auristatins; epothilones; daunorubicins and doxorubicins; alkylating agents, such as thiotepa and cyclophosphamide (CYTOXAN™); alkyl sulfonates such as busulfan, improsulfan and piposulfan; aziridines, such as benzodopa, carboquone, meturedopa, and uredopa; ethylenimines and methylamelamines including altretamine, triethylenemelamine, trietylene-phosphoramide, triethylenethiophosphaoramide and trimethylolomelamine; acetogenins (especially bullatacin and bullatacinone); camptothecins (including the synthetic analogue topotecan); bryostatin; callystatin; CC-1065 (including its adozelesin, carzelesin and bizelesin synthetic analogues); cryptophycins (particularly cryptophycin 1 and cryptophycin 8); duocarmycin (including the synthetic analogues, KW-2189 and CBI-TMI); eleutherobin; pancratistatin; sarcodictyins; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics, such as the enediyne antibiotics (e.g. calicheamicins, especially calicheamicin yl; dynemicin, including dynemicin A; esperamicin; as well as neocarzinostatin chromophore and related chromoprotein enediyne antiobiotic chromomophores), aclacinomysins, actinomycin, authramycin, azaserine, bleomycins, cactinomycin, carabicin, caminomycin, carzinophilin; chromomycins, dactinomycin, detorubicin, 6-diazo-5-oxo-L-norleucine, other doxorubicin derivatives including morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites, such as methotrexate and 5-fluorouracil (5-FU); folic acid analogues, such as denopterin, methotrexate, pteropterin, trimetrexate; purine analogs, such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-fluorouracil; androgens, such as calusterone, dromostanolone propionate, epitiostanol, mepitiostane, testolactone; anti-adrenals, such as aminoglutethimide, mitotane, trilostane; folic acid replenisher, such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; maytansinoids, such as maytansine, ansamitocins, DM-1, DM-4; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, RhonePoulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; capecitabine; anti-hormonal agents that act to regulate or inhibit hormone action on tumors, such as antiestrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens, such as flutamide, nilutamide, bicalutamide, leuprolide, and goserelin; siRNA; and pharmaceutically acceptable salts, acids or derivatives of any of the above as well as analogues and derivatives thereof, some of which are described below.

In one embodiment of the present invention, D is a dolastatin, auristatin, doxorubicin, epothilone, or any analogue or derivative thereof.

In one embodiment of the present invention, D is dolastatin or any derivative thereof.

In one embodiment of the present invention, D is dolastatin 15 or any derivative thereof.

In one embodiment of the present invention, D is auristatin F or any derivative thereof.

In one embodiment of the present invention, D is dolastatin 10, dolastatin 15, auristatin F or an aminoepothilone.

In one embodiment of the present invention, D is dolastatin 10.

In one embodiment of the present invention, D is dolastatin 15.

In one embodiment of the present invention, D is auristatin F.

Dolastatins that can be used in the present invention are well known in the art and can be isolated from natural sources according to known methods or prepared synthetically according to known methods.

Examples of suitable dolastatins include monomethyl and desmethyl dolastatins 10, 15, C, D and H, monomethyl and desmethyl isodolastatin H, and analogues and derivatives thereof. These dolastatins contain a primary or secondary amine at the N-terminus. Dolastatins 10 and 15 are the most potent cytotoxic agents among the naturally occurring dolastatins. Monomethyl and desmethyl dolastatins 10 and 15 can be prepared by chemical synthesis according to standard peptide synthesis chemistry.

Auristatins that can be used in the present invention include (but are not limited to) monomethyl and desmethyl auristatins E, F, EB, EFP, PY, PYE, PE, PHE, TP, 2-AQ and 6-AQ, e.g. described in U.S. Pat. No. 5,635,483; Int. J. Oncol. 15:367-72 (1999); Mol. Cancer Ther. 3:921-32 (2004); U.S. application Ser. No. 11/134,826; U.S. Patent Publication Nos. 20060074008 and 2006022925; and Pettit, G. R., et al. (2011) J. Nat. Prod. 74:962-8.

In one embodiment of the present invention, monomethyl and desmethyl auristatin and dolastatin 10 derivatives are represented by the formula:

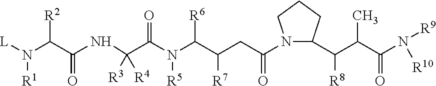

wherein L is either H, or may be understood as referring to the linker according to the present invention; R¹, R⁵ and R⁹ are each independently either H or C₁-C₅ alkyl; R², R³ and R⁶ are each independently either H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle or C₁-C₈ alkyl-(C₃-C₈ heterocycle); R⁴ is either H or CH₃; or R³ and R⁴ jointly form a carbocyclic ring with the carbon to which they are attached and have the formula —(CRₐR_{b})ₙ—, wherein Rₐ and R_{b} are independently selected from H, C₁-C₈ alkyl and C₃-C₈ carbocycle; and n is selected from 2, 3, 4, 5 and 6; R⁷ and R⁸ are each independently selected from H, OH, C₁-C₈ alkyl, C₃-C₈ carbocycle and O(C₁-C₈ alkyl); R¹⁰ is either CX₂—CX₂-aryl, CX₂—CX₂-(substituted aryl), CX₂—CX₂—(C₃-C₈ heterocycle), CX₂—(C₃-C₁₀ heterocycle), CX₂—CX₂—(C₃-C₈ carbocycle), C(═O)O(C₁-C₄ alkyl) or CH(CH₂R¹²)C(═O)ZR¹¹; each occurrence of X is independently either H, OH, C₁-C₈ alkyl, C₃-C₈ carbocycle, C₃-C₈ heterocycle, 2-thiazole or O(C₁-C₈ alkyl); Z is either O, S, NH or N(C₁-C₈ alkyl); R¹¹ is either H, C₁-C₂₀ alkyl, aryl, C₃-C₈ heterocycle, (R¹³O)ₘ—R¹⁴ or (R¹³O)ₘ—CH (R¹⁵)₂; R¹² is either aryl or C₃-C₈ heterocycle; m is an integer ranging from 1-1000; R¹³ is C₂-C₈ alkyl; R¹⁴ is H or C₁-C₈ alkyl; each occurrence of R¹⁵ is independently H, COOH, (CH₂)—N(R¹⁶)₂, (CH₂)—SO₃H or (CH₂)ₙ—SO₃—C₁-C₈ alkyl; each occurrence of R¹⁶ is independently H, C₁-C₈ alkyl or (CH₂)ₙ—COOH; and n is an integer in the range from 0 to 6.

In one embodiment of the present invention, monomethyl and desmethyl auristatins and dolastatin 10 derivatives are represented by the formula:

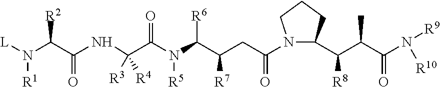

wherein the substituents are as described above.

In one embodiment of the present invention, monomethyl and desmethyl auristatins and dolastatin 10 derivatives are represented by the formula:

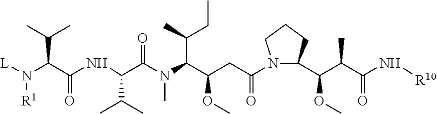

wherein the substituents are as described above.

In one embodiment of the present invention, monomethyl and desmethyl auristatin F derivatives are represented by the formula:

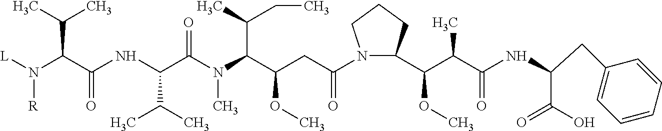

wherein L is either H, or may be understood as referring to the linker according to the present invention; and R is either H or CH₃.

In one embodiment of the present invention, monomethyl and desmethyl dolastatin 10 derivatives are represented by the formula:

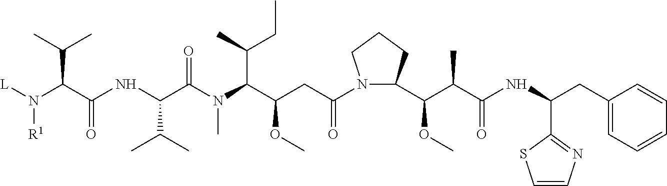

wherein L is either H, or may be understood as referring to the linker according to the present invention; and R¹ is either H or CH₃.

In one embodiment of the present invention, monomethyl and desmethyl dolastatin 15 analogues and derivatives are represented by the formula:

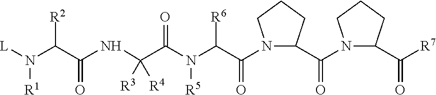

wherein L, R¹, R², R³, R⁴, R⁵ and R⁶ are as described above; R⁷ is either OH, NH₂, NHR⁸ or NR⁸R⁹; R⁸ and R⁹ are each independently either H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle, C₁-C₈ alkyl-(C₃-C₈ heterocycle), benzyl or tert-butyl; or R⁸ and R⁹ jointly form a heterocyclic ring with the nitrogen to which they are attached and have the formula —(CRₐR_{b})ₙ—, wherein Rₐ and R_{b} are independently selected from H, C₁-C₈ alkyl, C₃-C₈ carbocycle, aryl, C₁-C₈ alkyl-aryl, C₁-C₈ alkyl-(C₃-C₈ carbocycle), C₃-C₈ heterocycle, C₁-C₈ alkyl-(C₃-C₈ heterocycle), O(C₁-C₈ alkyl), a double bond with neighboring carbon atom, or they jointly form a carbonyl group; and n is selected from 2, 3, 4, 5 and 6.

In one embodiment of the present invention, monomethyl and desmethyl dolastatin 15 analogues and derivatives are represented by the formula:

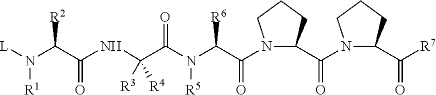

wherein the substituents are as described above.

In one embodiment of the present invention, the monomethyl or desmethyl dolastatin 15 analogue or derivative is selected from the group of monomethyl and desmethyl dolastatin 15, monomethyl and desmethyl cemadotin, monomethyl and desmethyl tasidotin, and monomethyl and desmethyl P5 (the corresponding dimethyl compounds are described in Bai et al. 2009. Mol. Pharmacol. 75:218-26).

In one embodiment of the present invention, monomethyl and desmethyl dolastatin 15 analogues and derivatives are represented by the formula:

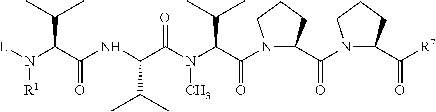

wherein the substituents are as described above.

In one embodiment of the present invention, monomethyl and desmethyl dolastatin 15 derivatives are represented by the formula:

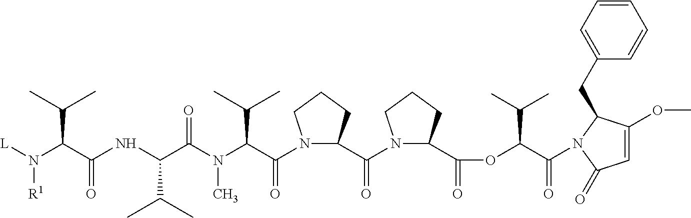

wherein L is either H, or may be understood as referring to the linker according to the present invention; and R¹ is either H or CH₃.

The cytotoxic agent according to the present invention may also be daunorubicin or doxorubicin. The primary amine group of the daunosamine moiety can be used, or daunorubicin or doxorubicin of the present invention can be modified to comprise another primary or secondary amine moiety. Preferred doxorubicin and daunorubicin payload molecules useful in the present invention are according to the formula:

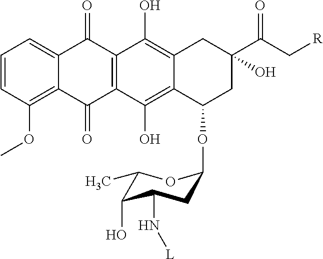

wherein R is either H or OH; and L is either H, or may be understood as referring to the linker according to the present invention.

In one embodiment of the present invention, D is an epothilone.

Epothilones that can be used in the present invention are well known in the art and can be isolated from natural sources according to known methods or prepared synthetically according to known methods (reviewed e.g. in The epothilones: An outstanding family of anti-tumor agents, eds. Kinghorn, A. D., et al. Springer-Verlag, Wien, 2009).

In one embodiment of the present invention, an epothilone is modified to contain a primary or secondary amine group for conjugation with a linker according to the invention.

In one embodiment of the present invention, the payload molecule is a 21-aminoepothilone according to the formula:

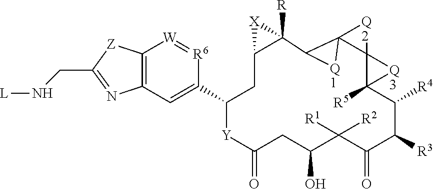

Wherein L is either H, or may be understood as referring to the linker according to the present invention; X is either O or a C—C bond (forming a double bond between C12 and C13 of the epothilone ring); Y is either O (lactone form) or NH (lactam/aza form); Z is either S or O; Q¹, Q² and Q³ are either absent or one of Q¹, Q² and Q³ is a C—C bond (forming a double bond between either C9 and C10, C8 and C9, or C7 and C8 of the epothilone ring, respectively); W is either absent or CH; R, R¹, R², R³ and R⁵ are each independently either H or CH₃; R⁴ is either H, OH or CH₂—CH═CH₂; and R⁶ is either H or CH₃ when W is absent, or CH when W is CH.

In some embodiments of the present invention, the epothilone is a 21-aminoepothilone selected from the group of 21-aminoepothilones A, B, C, D, E, F, A₁, A₂, A₈, A₉, C₁, C₂, C₃, C₄, C₅, C₆, C₇, C₈, C₉, D₁, D₂, D₅, G₁, G₂, H₁, H₂, I₁, I₂, I₃, I₄, I₅, I₆ and K; 21-amino-trans-epothilones C₁ and C₂; 21-amino modifications of ixabepilone, patupilone, sagopilone and (E)-9,10-didehydroepothilone D; 21-aminomethylepothilone B (aminoepothilone B₁₀); and 20-aminomethyltioepothilone B (amino modification of ABJ879).

In one embodiment of the present invention, the epothilone is 21-aminoepothilone B, wherein X, Y═O, Z═S, Q¹, Q², Q³, W=absent, R, R¹, R², R³, R⁵, R⁶═CH₃ and R⁴═OH; and the structure is according to the formula:

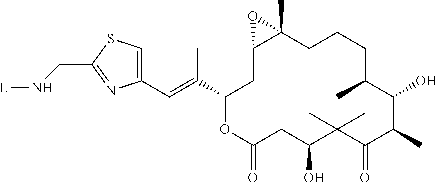

wherein L is either H, or may be understood as referring to the linker according to the present invention.

In one embodiment of the present invention, D is an aminoepothilone or any derivative thereof.

In one embodiment of the present invention, D is 21-aminoepothilone.

One skilled in the art of cytotoxic agents will readily understand that each of the cytotoxic agents described herein can be modified in such a manner that the resulting compound still retains the specificity and/or activity of the starting compound. The skilled person will also understand that many of these compounds can be used in place of the cytotoxic agents described herein. Thus, the cytotoxic agents of the present invention should be understood as including any analogues and derivatives of the compounds described herein.

In one embodiment of the present invention, R₁, R₂, R₃, R₄ and R₇ are each H; W is H; a is 1; b is 1; c and e are each 0; and d is 4.

In one embodiment of the present invention, R₃, R₄, and R₇ are each H; W is H; b is 1; a, c and e are each 0; and d is 4.

In one embodiment of the present invention, the linker-payload molecule conjugate is represented by formula I, wherein X is azide; Y is an oxygen; Z is absent; D is monomethylauristatin F or dolastatin 10; R₁, R₂, R₃, R₄ and R₇ are each H; W is H; a is 1; b is 1; c and e are each 0; and d is 4.

In one embodiment of the present invention, the linker-payload molecule conjugate is represented by formula I, wherein X is an alkyne; Y is an oxygen; Z is absent; D is monomethylauristatin F or dolastatin 10; R₁, R₂, R₃, R₄ and R₇ are each H; W is H; a is 1; b is 1; c and e are each 0; and d is 4.

In one embodiment of the present invention, the linker-payload molecule conjugate is represented by formula I, wherein X is an alkyne; Y is an oxygen; Z is absent; D is monomethylauristatin F; R₁, R₂, R₃, R₄ and R₇ are each H; W is H; a is 1; b is 1; c and e are each 0; and d is 4.

In one embodiment of the present invention, the linker-payload molecule conjugate is represented by formula I, wherein X is CH≡C; Y is an oxygen; Z is absent; D is monomethylauristatin F; R₁, R₂, R₃, R₄ and R₇ are each H; W is H; a is 1; b is 1; c and e are each 0; and d is 4.

In one embodiment of the present invention, the linker-payload molecule conjugate is represented by formula I, wherein X is an alkyne; Y is absent; Z is absent; D is monomethylauristatin F or dolastatin 10; R₃, R₄, and R₇ are each H; W is H; b is 1; a, c and e are each 0; and d is 4.

In one embodiment of the present invention, the linker-payload molecule conjugate is represented by formula I, wherein X is CH≡C; Y is absent; Z is absent; D is monomethylauristatin F or dolastatin 10; R₃, R₄, and R₇ are each H; W is H; b is 1; a, c and e are each 0; and d is 4.

In one embodiment of the present invention, the linker-payload molecule conjugate is represented by formula I, wherein X is azide; Y is absent; Z is absent; D is monomethylauristatin F or dolastatin 10; R₃, R₄, and R₇ are each H; W is H; b is 1; a, c and e are each 0; and d is 4.

In one embodiment of the present invention, the linker-payload molecule conjugate is represented by formula I, wherein X is azide; Y is absent; Z is absent; D is monomethylauristatin F; R₃, R₄, and R₇ are each H; W is H; b is 1; a, c and e are each 0; and d is 4.

In one embodiment of the present invention, the linker-payload molecule conjugate is represented by formula I, wherein X is azide; Y is an oxygen; Z is absent; D is monomethylauristatin F or dolastatin 10; R₁, R₂, R₃, R₄, and R₇ are each H; W is H; a is 1; b is 1; c and e are 0; and d is 4.

In one embodiment of the present invention, the linker-payload molecule conjugate is represented by formula I, wherein X is an alkyne; Y is an oxygen; Z is absent; D is monomethylauristatin F or dolastatin 10; R₁, R₂, R₃, R₄, and R₇ are each H; W is H; a is 1; b is 1; c and e are 0; and d is 4.

In one embodiment of the present invention, the linker-payload molecule conjugate is represented by formula I, wherein X is an alkyne; Y is an oxygen; Z is absent; D is dolastatin 10; R₁, R₂, R₃, R₄, and R₇ are each H; W is H; a is 1; b is 1; c and e are 0; and d is 4.

In one embodiment of the present invention, the linker-payload molecule conjugate is represented by formula I, wherein X is CH≡C; Y is an oxygen; Z is absent; D is dolastatin 10; R₁, R₂, R₃, R₄, and R₇ are each H; W is H; a is 1; b is 1; c and e are 0; and d is 4.

In one embodiment of the present invention, the linker-payload molecule conjugate is represented by formula I, wherein X is azide; Y is absent; Z is absent; D is monomethylauristatin F or dolastatin 10; R₃, R₄, and R₇ are each H; W is H; b is 1; a, c and e are each 0; and d is 4.

In one embodiment of the present invention, the linker-payload molecule conjugate is represented by formula I, wherein X is azide; Y is absent; Z is absent; D is dolastatin 10; R₃, R₄, and R₇ are each H; W is H; b is 1; a, c and e are each 0; and d is 4.

In one embodiment of the present invention, the linker-payload molecule conjugate is represented by formula I, wherein X is azide; Y is an oxygen; Z is absent; D is monomethylauristatin F or dolastatin 10; R₁, R₂, R₃, R₄, and R₇ are each H; W is H; a is 1; b is 1; c and e are each 0; and d is 4.

In one embodiment of the present invention, the linker-payload molecule conjugate is represented by formula I, wherein X is an alkyne; Y is an oxygen; Z is absent; D is monomethylauristatin F or dolastatin 10; R₁, R₂, R₃, R₄, and R₇ are each H; W is H; a is 1; b is 1; c and e are each 0; and d is 4.

In one embodiment of the present invention, the linker-payload molecule conjugate is
N-(6-propargyl-D-galactosyl)-monomethylauristatin F,
N-(6-azido-D-galactosyl)-monomethylauristatin F,
N-(6-propargyl-D-galactosyl)-dolastatin 10, or
N-(6-azido-D-galactosyl)-dolastatin 10.

In one embodiment of the present invention, the linker-payload molecule conjugate is
N-(2-deoxy-D-glucosyl)-monomethylauristatin F,
N-[6-O-(β-D-galacto-pyranosyl)-D-galactosyl]-monomethylauristatin F,
N-[4-O-(β-D-galactopyranosyl)-D-glucosyl]-monomethylauristatin F,
N-[2-acetamido-2-deoxy-4-O-(β-D-galactopyranosyl)-D-glucosyl)-monomethylauristatin F,
N-{4-O-[4-O-(α-D-galactopyranosyl)-β-D-galactopyranosyl]-D-glucosyl}-monomethylauristatin F, or
N-{6-O-[3-O-(α-N-acetylneuraminyl)-β-D-galactopyranosyl]-D-galactosyl}-monomethylauristatin F.

In one embodiment of the present invention, the linker-payload molecule conjugate is N-(propargylgalactose)aminoepothilone or N-(2-deoxyglucosyl)aminoepothilone.

In one embodiment of the present invention, the linker-payload molecule conjugate is represented by formula V, wherein Z is OH; D is monomethylauristatin F or dolastatin 10; R₃, R₄, and R⁷ are each H; W is H; b is 1; c is 0; e is 1; and d is 3.

In one embodiment of the present invention, the linker-payload molecule conjugate is represented by formula V, wherein Z is OH; D is monomethylauristatin F; R₃, R₄, and R⁷ are each H; W is H; b is 1; c is 0; e is 1; and d is 3.

In one embodiment of the present invention, the linker-payload molecule conjugate is represented by formula V, wherein Z is a saccharide; D is monomethylauristatin F or dolastatin 10; R₃, R₄, and R⁷ are each H; W is H; b is 1; c and e are each 0; and d is 4.

In one embodiment of the present invention, the linker-payload molecule conjugate is represented by formula V, wherein Z is a saccharide; D is monomethylauristatin F; R₃, R₄, and R⁷ are each H; W is H; b is 1; c and e are each 0; and d is 4.

In one embodiment of the present invention, the linker-payload molecule conjugate is represented by formula V, wherein Z is β-D-galactopyranosyl; D is monomethylauristatin F; R₃, R₄, and R⁷ are each H; W is H; b is 1; c and e are each 0; and d is 4.

In one embodiment of the present invention, the linker-payload molecule conjugate is represented by formula V, wherein Z is β-D-galactopyranosyl; D is monomethylauristatin F; R₃ is a substituted alkyl; R₄ and R⁷ are each H; W is H; b is 1; c and e are each 0; and d is 4.

In one embodiment of the present invention, the linker-payload molecule conjugate is represented by formula V, wherein Z is β-D-galactopyranosyl; D is monomethylauristatin F; R₃ is 1,2-dihydroxyethyl; R₄ and R⁷ are each H; W is H; b is 1; c and e are each 0; and d is 4.

In one embodiment of the present invention, the linker-payload molecule conjugate is represented by formula V, wherein Z is β-D-galactopyranosyl; D is monomethylauristatin F; R₃, R₄, and R₇ are each H; W is H; b is 1; c and e are each 0; and d is 4.

In one embodiment of the present invention, the linker-payload molecule conjugate is represented by formula IV, wherein D is dolastatin 10; a is 1, 2, 3 or 4; and R₁ and R₂ are each H.

In one embodiment of the present invention, the linker-payload molecule conjugate is represented by formula IV, wherein D is dolastatin 15; a is 1, 2, 3 or 4; and R₁ and R₂ are each H.

In one embodiment of the present invention, the linker-payload molecule conjugate is
N-(3-butynyl)-dolastatin 10 or
N-(4-pentynyl)-dolastatin 10.

In one embodiment of the present invention, the linker-payload molecule conjugate is
N-(3-butynyl)-dolastatin 15 or
N-(4-pentynyl)-dolastatin 15.

In one embodiment of the present invention, the linker-payload molecule conjugate is
N-(3-butynyl)-monomethylauristatin F or
N-(4-pentynyl)-monomethylauristatin F.

The present invention also relates to a method for preparing a linker-payload molecule conjugate or a cell binder-linker-payload molecule conjugate according to the present invention, comprising the steps of:

a) preparing an activated linker comprising i) a group capable of forming a secondary or tertiary amine with an amine group of a payload molecule, and optionally ii) a group capable of reaction with a cell binder, and/or optionally iii) one or more hydroxyl groups;

b) reacting the activated linker of step a) with a payload molecule to form a secondary or tertiary amine;

optionally c) modifying a cell binder so as to comprise functional group capable of reacting with the activated linker of step b); and

optionally d) reacting the activated linker of step b) with a cell binder optionally modified in step c).

In one embodiment of the present invention, the activated linker comprises i) a moiety or portion of a molecule represented by any one of formulas I, II, III, IV or V that does not comprise the payload molecule D, or wherein a molecule is represented by formula IV, the moiety or portion of the molecule that does not comprise the payload molecule D and the cell binder B, and ii) a functional group capable of forming a secondary or tertiary amine with an amine group of a payload molecule.

In one embodiment of the present invention, the group capable of reaction with a cell binder is X as defined above.

In one embodiment of the present invention, the group capable of forming a secondary or tertiary amine with an amine group of a payload molecule is an aldehyde, ketone, or alkyl halide.

The synthetic routes to produce the linkers, linker-payload molecule conjugates and cell binder-linker-payload molecule conjugates of the present invention are shown in Examples and Figures. The linkers comprise three elements: a) a group, such as a N-hydroxysuccimimide ester, maleimido group, haloacetyl group, hydrazide, azide, alkyne, aldehyde, ketone or hydroxylamine capable of reaction with a cell binder, b) a group, such as but not limited to, an aldehyde, ketone, or alkyl halide, capable of forming a secondary or tertiary amine with an amine group of a payload molecule, and optionally c) hydroxyl group(s) within a hydrophilic linker element. The hydroxyl substituents can be introduced by methods described herein. For example, a reducing sugar comprising hydroxyl groups can be reductively aminated to a primary or secondary amine group of a payload molecule. Alternatively, an alkyl chain can be introduced to the amine by for example as shown in shown in Examples. First the desired reactive group, such as but not limited to, azide or alkyne is introduced to the reducing sugar or the alkyl chain by the reactions shown in Examples.

The present invention also relates to a pharmaceutical composition comprising an effective amount of the linker-payload molecule conjugate or the cell binder-linker-payload molecule conjugate according to the invention and a pharmaceutically acceptable carrier.

The “therapeutically effective amount” or “effective amount” of the linker-payload molecule conjugates described herein refer to the dosage regimen for modulating the growth of cancer cells and/or treating a patient's disease, and is selected in accordance with a variety of factors, including the age, weight, sex, diet and medical condition of the patient, the severity of the disease, the route of administration, and pharmacological considerations, such as the activity, efficacy, pharmacokinetic and toxicology profiles of the particular compound used. The “therapeutically effective amount” can also be determined by reference to standard medical texts, such as the Physicians Desk Reference 2004. The patient is preferably an animal, more preferably a mammal, most preferably a human. The patient can be male or female, and can be an infant, child or adult.

In one embodiment of the present invention, the pharmaceutical composition comprises a composition for e.g. oral, parenteral, transdermal, intraluminal, intraarterial, intrathecal and/or intranasal administration or for direct injection into tissue. Administration of the pharmaceutical composition may be effected in different ways, e.g. by intravenous, intraperitoneal, subcutaneous, intramuscular, topical or intradermal administration. The pharmaceutical composition of the present invention may further comprise a pharmaceutically acceptable carrier. Examples of suitable pharmaceutically acceptable carriers are well known in the art and include e.g. phosphate buffered saline solutions, water, oil/water emulsions, wetting agents, and liposomes. Compositions comprising such carriers may be formulated by methods well known in the art. Dosages and dosage regimens, as known in the art, may vary depending on a number of factors and may be determined depending on e.g. the patient's age, size, the nature of the conjugate, and the administration route. The pharmaceutical composition may further comprise other components such as vehicles, additives, preservatives, other pharmaceutical compositions administrated concurrently, and the like.

In one embodiment of the present invention growth of selected cell populations is modulated by contacting the pharmaceutical composition with said cell populations.

The present invention also relates to a method for modulating growth of a cell population, comprising the step of contacting the linker-payload conjugate or cell binder-linker-payload conjugate according to the invention or the pharmaceutical composition according to the invention with the cell population.

In this context, the term “a cell population” should be understood as referring to one or more cell populations.

The linker-payload molecule conjugates of the present invention can be contacted in vitro, in vivo and/or ex vivo to modulate the growth of a cell population, for example, cancer cells, including, for example, cancer of the blood, plasma, lung, breast, colon, prostate, kidney, pancreas, brain, bones, ovary, testes, and lymphatic organs; more preferably lung, colon prostrate, plasma, blood or colon cancer; or in autoimmune diseases, such as systemic lupus, rheumatoid arthritis, and multiple sclerosis; graft rejections, such as renal transplant rejection, liver transplant rejection, lung transplant rejection, cardiac transplant rejection, and bone marrow transplant rejection; graft versus host disease; viral infections, such as CMV infection, HIV infection, and AIDS; and parasite infections, such as giardiasis, amoebiasis, schistosomiasis, and the like; or, for example, low density lipoprotein receptor-related protein-1 LRP-1 expressing cells such as fibrosarcoma cells. “Modulating the growth of selected cell populations” includes inhibiting the proliferation of said cells from dividing to produce more cells; reducing the rate of increase in cell division as compared, for example, to untreated cells; killing selected cell populations; and/or preventing selected cell populations (such as cancer cells) from metastasizing. The growth of selected cell populations can be modulated in vitro, in vivo or ex vivo.

In one embodiment of the present invention, the pharmaceutical composition comprises an effective amount of the cell binder-linker-payload molecule conjugate according to the invention and a pharmaceutically acceptable carrier.

In one embodiment of the present invention, the pharmaceutical composition comprises an effective amount of the cell binder-linker-payload molecule conjugate wherein the cell binder is the antibody cetuximab and a pharmaceutically acceptable carrier.

In one embodiment of the present invention, the pharmaceutical composition comprises an effective amount of the cell binder-linker-payload molecule conjugate wherein the cell binder is the antibody trastuzumab and a pharmaceutically acceptable carrier.

In one embodiment of the present invention, the pharmaceutical composition comprises an effective amount of the cell binder-linker-payload molecule conjugate wherein the cell binder is the antibody panitumumab and a pharmaceutically acceptable carrier.

In one embodiment of the present invention, the pharmaceutical composition comprises an effective amount of the cell binder-linker-payload molecule conjugate wherein the cell binder is the antibody rituximab and a pharmaceutically acceptable carrier.

In one embodiment of the present invention, the pharmaceutical composition comprises an effective amount of the cell binder-linker-payload molecule conjugate wherein the cell binder is the antibody bevacizumab and a pharmaceutically acceptable carrier.

In one embodiment of the present invention, the pharmaceutical composition comprises an effective amount of the cell binder-linker-payload molecule conjugate wherein the cell binder is the antibody tositumomab, etanercept, adalimumab, alemtuzumab, gemtuzumab ozogamicin, efalizumab, rituximab, infliximab, abciximab, basiliximab, palivizumab, omalizumab, daclizumab, epratuzumab, lintuzumab, nimotuzumab, 2G12 or ibritumomab tiuxetan, and a pharmaceutically acceptable carrier.

In one embodiment, the cell population is a cancer cell population.

The present invention further relates to the linker-payload molecule conjugate or the cell binder-linker-payload molecule conjugate according to one or more embodiments of the invention for use as a medicament.

The present invention further relates to the linker-payload molecule conjugate or the cell binder-linker-payload molecule conjugate according to one or more embodiments of the invention for use in therapy.

The present invention further relates to the linker-payload molecule conjugate or the cell binder-linker-payload molecule conjugate according to one or more embodiments of the invention for use in the treatment of cancer.

The present invention further relates to the linker-payload molecule conjugate or the cell binder-linker-payload molecule conjugate according to one or more embodiments of the invention for the manufacture of a medicament.

The present invention further relates to the linker-payload molecule conjugate or the cell binder-linker-payload molecule conjugate according to one or more embodiments of the invention for the manufacture of a medicament for the treatment of cancer.

In one embodiment, the cancer is selected from the group consisting of leukemia, lymphoma, breast cancer, prostate cancer, ovarian cancer, colorectal cancer, gastric cancer, squamous cancer, small-cell lung cancer, head-and-neck cancer, and testicular cancer, including a metastatic, advanced or drug-resistant, or multidrug resistant, version thereof.

The present invention further relates to a method of treating and/or modulating the growth of and/or prophylaxis of tumour cells in humans or animals, wherein the linker-payload molecule conjugate, the cell binder-linker-payload molecule or the pharmaceutical composition according to one or more embodiments of the invention is administered to a human or animal in an effective amount.

In one embodiment, the tumour cells are selected from the group consisting of leukemia cells, lymphoma cells, breast cancer cells, prostate cancer cells, ovarian cancer cells, colorectal cancer cells, gastric cancer cells, squamous cancer cells, small-cell lung cancer cells, head-and-neck cancer cells, and testicular cancer cells, or metastatic, or drug-resistant, or multidrug resistant, versions thereof.

The present invention further relates to a method of treating cancer in humans or animals, wherein the linker-payload molecule conjugate or the cell binder-linker-payload molecule conjugate according to one or more embodiments of the invention is administered to a human or animal in an effective amount.

In one embodiment, the linker-payload molecule conjugate, the cell binder-linker-payload molecule or a pharmaceutical composition according to one or more embodiments of the invention can also be used to effectively treat drug resistant tumours, including multidrug resistant tumours, “multidrug resistance” meaning the resistance of tumor cells to more than one chemotherapeutic agent. Multidrug resistance may be aided e.g. by a P-glycoprotein transmembrane pump that lowers the concentration of drugs in the cell. As is known in the art, the resistance of cancer cells to chemotherapy is one of the central problems in the management of cancer. Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of a linker-payload molecule conjugate, a cell binder-linker-payload molecule or a pharmaceutical composition according to one or more embodiments of the invention in the treatment of these “hormone-resistant” or “hormone-refractory” cancers.

In one embodiment, the linker-payload molecule conjugate, the cell binder-linker-payload molecule or a pharmaceutical composition according to one or more embodiments of the invention, is used in the treatment of metastatic, advanced, drug- or hormone-resistant, or multidrug resistant, versions of solid tumours. In one embodiment, the linker-payload molecule conjugate, the cell binder-linker-payload molecule or a pharmaceutical composition according to one or more embodiments of the invention is used in the treatment of a leukemia, including a metastatic, advanced or drug-resistant, or multidrug resistant, version thereof.

The embodiments of the invention described hereinbefore may be used in any combination with each other. Several of the embodiments may be combined together to form a further embodiment of the invention. A product, or a use, or a method to which the invention is related, may comprise at least one of the embodiments of the invention described hereinbefore.

### EXAMPLES

In the following, the present invention will be described in more detail. Reference will now be made in detail to the embodiments of the present invention, examples of which are illustrated in the accompanying drawings. The description below discloses some embodiments of the invention in such detail that a person skilled in the art is able to utilize the invention based on the disclosure. Not all steps of the embodiments are discussed in detail, as many of the steps will be obvious for the person skilled in the art based on this specification.

### Example 1

### Synthesis of Dolastatin Derivatives

Unless otherwise noted, materials were obtained from commercial suppliers in the highest purity grade available and used without further purifications. Reaction solvents were dried and distilled prior to use when necessary. All reactions containing moisture- or air-sensitive reagents were carried out under an argon atmosphere. Monomethylauristatin F (MMAF) and monomethyldolastatin 10 were purchased from Concortis (San Diego, Calif., USA). Sodium cyanoborohydride, sodium hydride (NaH), methanol, 4-bromo-1-butyne, 5-iodo-1-pentyne, 2-deoxy-D-glucose, 6-O-(β-D-galacto-pyranosyl)-D-galactose, diisopropylethylamine and 2,5-dihydroxybenzoic acid were purchased from Sigma-Aldrich. Dimethylsuphoxide (DMSO) and N,N-dimethylformamide (DMF) were purchased from VWR. 2-acetamido-2-deoxy-4-O-(β-D-galactopyranosyl)-D-glucose, N-{4-O-[4-O-(α-D-galactopyranosyl)-β-D-galactopyranosyl]-D-glucose and 4-O-[3-O-(α-N-acetyl-neuraminyl)-β-D-galactopyranosyl]-D-glucose were from Kyowa Hakko Kogyo. Trifluoroacetic acid and ammonium hydrogen carbonate were purchased from Fluka, acetonitrile (ACN) from J. T. Baker and disuccinimidyl glutarate from Pierce.

The NMR spectra were recorded with a Bruker Avance spectrometer operating at 600.13 MHz (¹H: 600.13 MHz, ¹³C: 150.90 MHz). Pulse sequences provided by the manufacturer were utilized. The probe temperature during the experiments was kept at 22° C. unless otherwise mentioned. Chemical shifts are expressed on the δ scale (in ppm) using TMS (tetramethylsilane), residual chloroform, acetone, H₂O or methanol as internal standards. Coupling constants are given in Hz and provided only once when first encountered. Coupling patterns are given as s, singlet, d, doublet, t, triplet etc.

TLC was performed on aluminium sheets precoated with silica gel 60 F₂₅₄ (Merck). Flash chromatography was carried out on silica gel 60 (0.040-0.060 mm, Aldrich). Spots were visualized by UV followed by charring with 1:8 H₂SO₄/MeOH and heating.

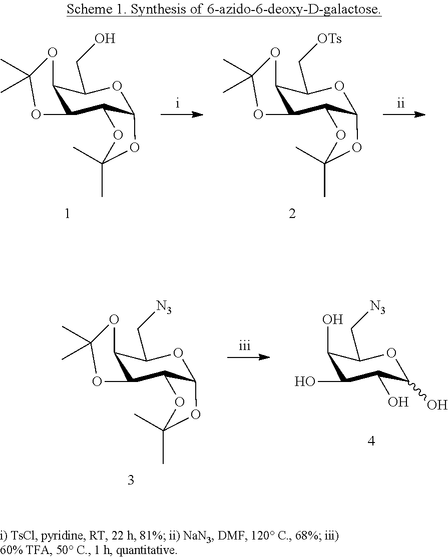

Synthesis of 1,2;3,4-di-O-isopropylidene-6-O-tosyl-α-D-galactopyranose (Scheme 1.2): 0.39 g (1.5 mmol) of (Scheme 1.1) was dissolved in 5 ml of dry pyridine under an argon atmosphere. The reaction mixture was cooled on an ice bath and 0.88 g (3.1 equiv.) of TsCl was added. The reaction was slowly warmed to RT and stirred overnight. After 22 hours the reaction was diluted with 30 ml of CH₂Cl₂ and washed with 30 ml of ice-cold water. The organic phase was washed with 20 ml of 10% (w/v) aqueous CuSO₄-solution, 20 ml of saturated NaHCO₃-solution and 20 ml H₂O . The organic phase was separated, dried over Na₂SO₄, filtered and concentrated. The crude product was purified by column chromatography (Hexane:EtOAc 1:1) to give (Scheme 1.2) as a yellowish oil (0.49 g, 81%). TLC: R_{f}=0.74 (Hexane:EtOAc 1:1). ¹H NMR (600 MHz, CDCl₃, 22° C.): δ=7.81-7.32 (m, 4H, CH₃C₆H₄SO₂), 5.45 (d, 1H, J_{1,2}=4.9 Hz, H-1), 4.59 (dd, 1H, J_{3,2}=2.5, J_{3,4}=7.9 Hz, H-3), 4.29 (dd, 1H, H-2), 4.22-4.18 (m, 2H, H-6a, H-4), 4.09 (dd, 1H, J_{6b,5}=6.9, J_{6b,6a}=−10.3 Hz, H-6b), 4.05 (ddd, 1H, J_{5,4}=1.9, J_{5,6a}=6.2 Hz, H-5), 2.44 (s, 3H, CH₃C₆H₄SO₂), 1.50, 1.34, 1.31 and 1.28 (each s, each 3H, O₂C(CH₃)₂) ppm.

Synthesis of 1,2;3,4-di-O-isopropylidene-6-deoxy-6-azido-α-D-galactopyranose (Scheme 1.3). To a solution containing 1.5 g (3.7 mmol) of (Scheme 1.2) in 20 ml dry DMF (under an argon atmosphere) was added 1.7 g (7 equiv.) NaN₃ and the resulting mixture was stirred at 120° C. overnight. After 18 hours, the reaction mixture was brought to RT, diluted with 20 ml CHCl₃, filtered and concentrated. The crude product was purified by column chromatography (Hexane:EtOAc 3:1) to give (Scheme 1.3) as a colorless oil (0.7 g, 68%). TLC: R_{f}=0.52 (Hexane:EtOAc 3:1). ¹H NMR (600 MHz, CDCl₃, 22° C.): δ=5.55 (d, 1H, J_{1,2}=5.1 Hz, H-1), 4.63 (dd, 1H, J_{3,2}=2.5, J_{3,4}=8.1 Hz, H-3), 4.33 (dd, 1H, H-2), 4.19 (dd, 1H, J_{4,5}=2.0 Hz, H-4), 3.92 (ddd, 1H, J_{5,6b}=5.3, J_{5,6a}=7.8 Hz, H-5), 3.51 (dd, 1H, J_{6a,6b}=−12.9 Hz, H-6a), 3.36 (dd, 1H, H-6b), 1.55, 1.46, 1.35 and 1.34 (each s, each 3H, O₂C(CH₃)₂) ppm.

Synthesis of 6-azido-6-deoxy-D-galactose (Scheme 1.4). 80 mg (0.3 mmol) of (Scheme 1.3) was dissolved in 3 ml 60% TFA and the resulting mixture was stirred at 50° C. for 1 hour. The mixture was then diluted with water and concentrated to give (Scheme 1.4) as a colorless oil (60 mg, quantitative, furanose:pyranose 3:97, alpha_{pyranose}:beta_{pyranose} 35:65). Selected NMR-data: ¹H NMR (600 MHz, D₂O, 22° C.): δ=5.28 (d, 1H, J_{1,2}=4.7 Hz, H-1_{furanose}), 5.26 (d, 1H, J_{1,2}=3.9 Hz, H-1α_{pyranose}), 5.22 (d, 1H, J_{1,2}=3.4 Hz, H-1_{furanose}), 4.60 (d, 1H, J_{1,2}=7.8 Hz, H-1β_{pyranose}).

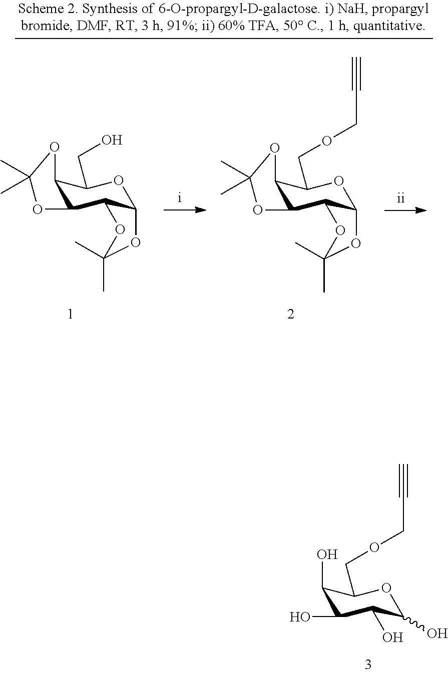

1,2;3,4-di-O-isopropylidene-6-O-propargyl-α-D-galactopyranose (Scheme 2.2). To a solution containing 0.27 g (1.0 mmol) 1 in 5 ml dry DMF (under an argon atmosphere) was added 75 mg (2.0 equiv.) NaH at 0° C. The resulting mixture was stirred for 20 min. and 171 μl (1.5 equiv.) of propargyl bromide was added. After 20 min. the mixture was brought to RT and stirred for an additional 2.5 hours. The mixture was cooled on an ice bath and quenched by the addition of MeOH (0.5 ml). The reaction mixture was brought to RT, diluted with 20 ml CH₂Cl₂ and washed with 20 ml saturated NaHCO₃-solution. The water phase was extracted with 20 ml CH₂Cl₂. The combined organic phase was washed with 20 ml H₂O , dried over Na₂SO₄, filtered and concentrated. The crude product was purified by column chromatography (Hexane:EtOAc 2:1) to give (Scheme 2.2) as a white solid (0.27 g, 91%). TLC: R_{f}=0.77 (Hexane:EtOAc 1:1). ¹H NMR (600 MHz, CDCl₃, 22° C.): δ=5.54 (d, 1H, J_{1,2}=5.1 Hz, H-1), 4.61 (dd, 1H, J_{3,2}=2.5, J_{3,4}=8.0 Hz, H-3), 4.32 (dd, 1H, H-2), 4.26 (dd, 1H, J_{4,5}=1.9 Hz, H-4), 4.25 (dd, 1H, J_{CH2a,≡CH}=2.4, J_{CH2a,≡CH2b}=−15.9 Hz, CH₂ₐC≡CH), 4.20 (dd, 1H, J_{CH2b,≡CH}=2.4 Hz, CH2bC≡CH), 4.00 (ddd, 1H, J_{5,6a}=5.4, J_{5,6b}=7.1 Hz, H-5), 3.78 (dd, 1H, J_{6a,6b}=−10.1 Hz, H-6a), 3.67 (dd, 1H, H-6b), 2.43 (dd, 1H, CH2C≡CH), 1.55, 1.45, 1.34 and 1.33 (each s, each 3H, O₂C(CH₃)₂) ppm.

Synthesis of 6-O-propargyl-D-galactose (Scheme 2.3). 25 mg (0.08 mmol) of (Scheme 2.3) was dissolved in 3 ml 60% TFA and the resulting mixture was stirred at 50° C. for 1 hour. The mixture was then diluted with water and concentrated to give (Scheme 2.3) as a colorless oil (18 mg, quantitative, furanose:pyranose 3:97, alpha_{pyranose}:beta_{pyranose} 35:65). Selected NMR-data: ¹H NMR (600 MHz, D₂O, 22° C.): δ=5.26 (d, 1H, J_{1,2}=4.7 Hz, H-1_{furanose}), 5.23 (d, 1H, J_{1,2}=3.8 Hz, H-1α_{pyranose}), 5.20 (d, 1H, J_{1,2}=3.5 Hz, H-1_{furanose}), 4.55 (d, 1H, J_{1,2}=7.9 Hz, H-1β_{pyranose}).

The following MMAF (1) and monomethyldolastatin 10 (2) derivatives (3-14) were prepared:

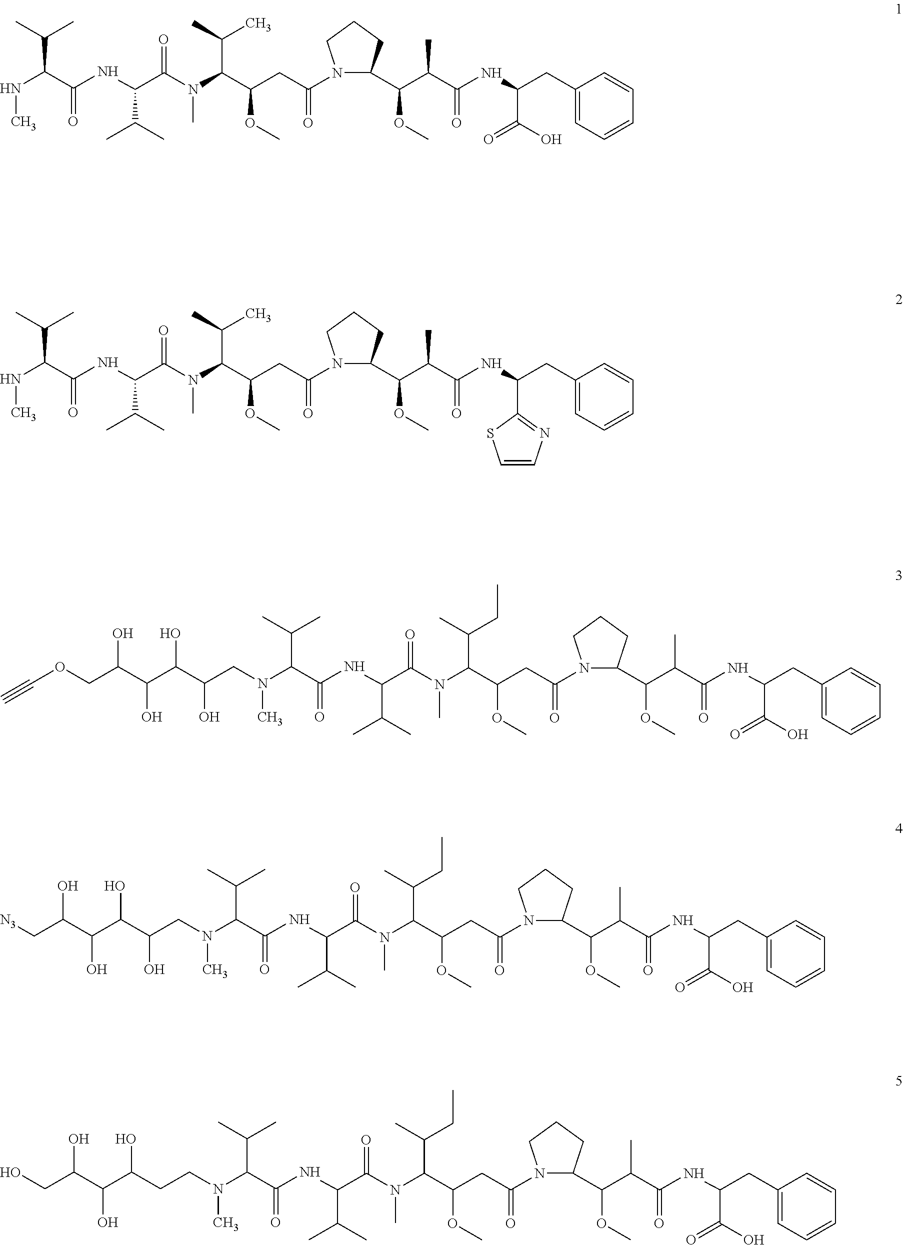

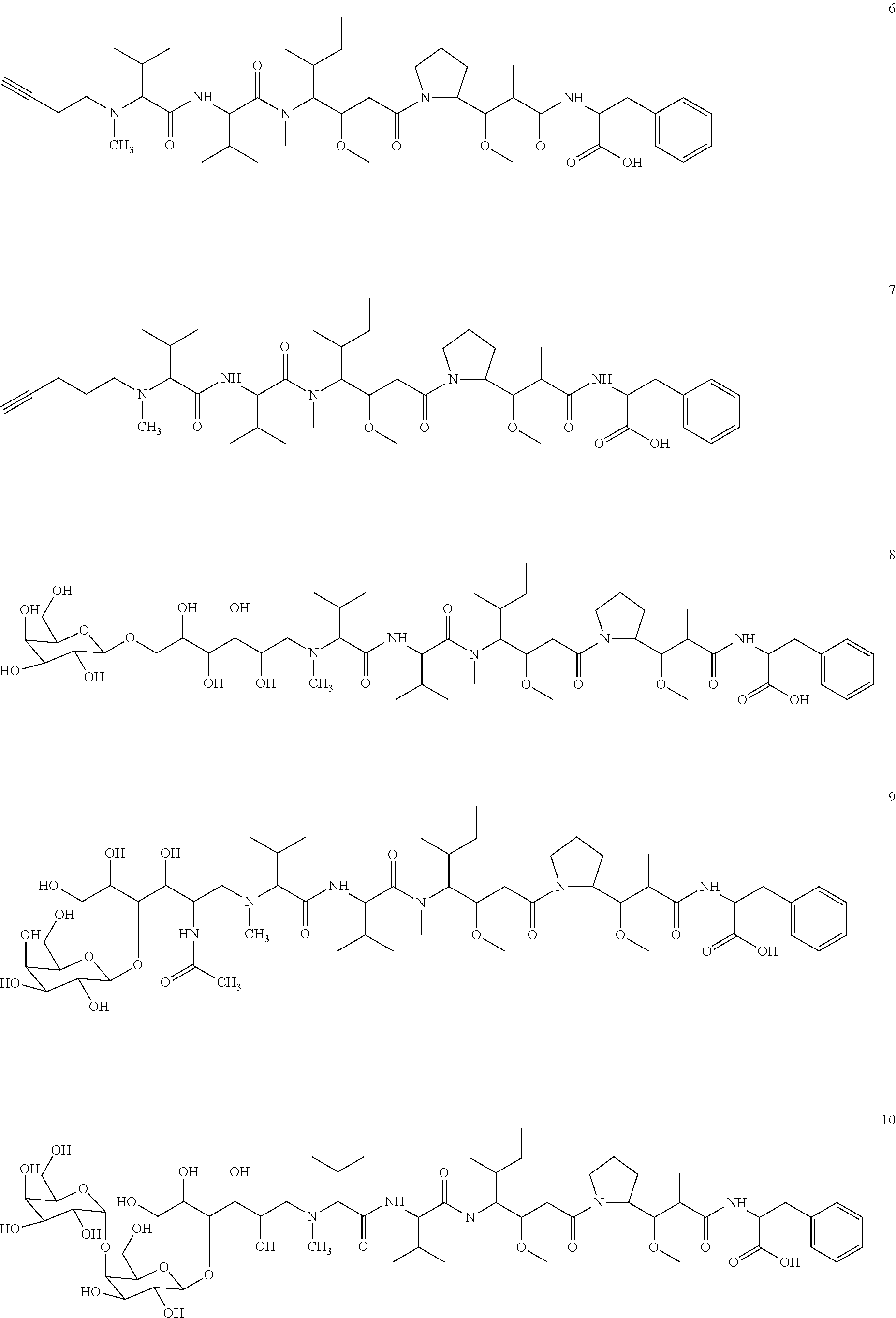

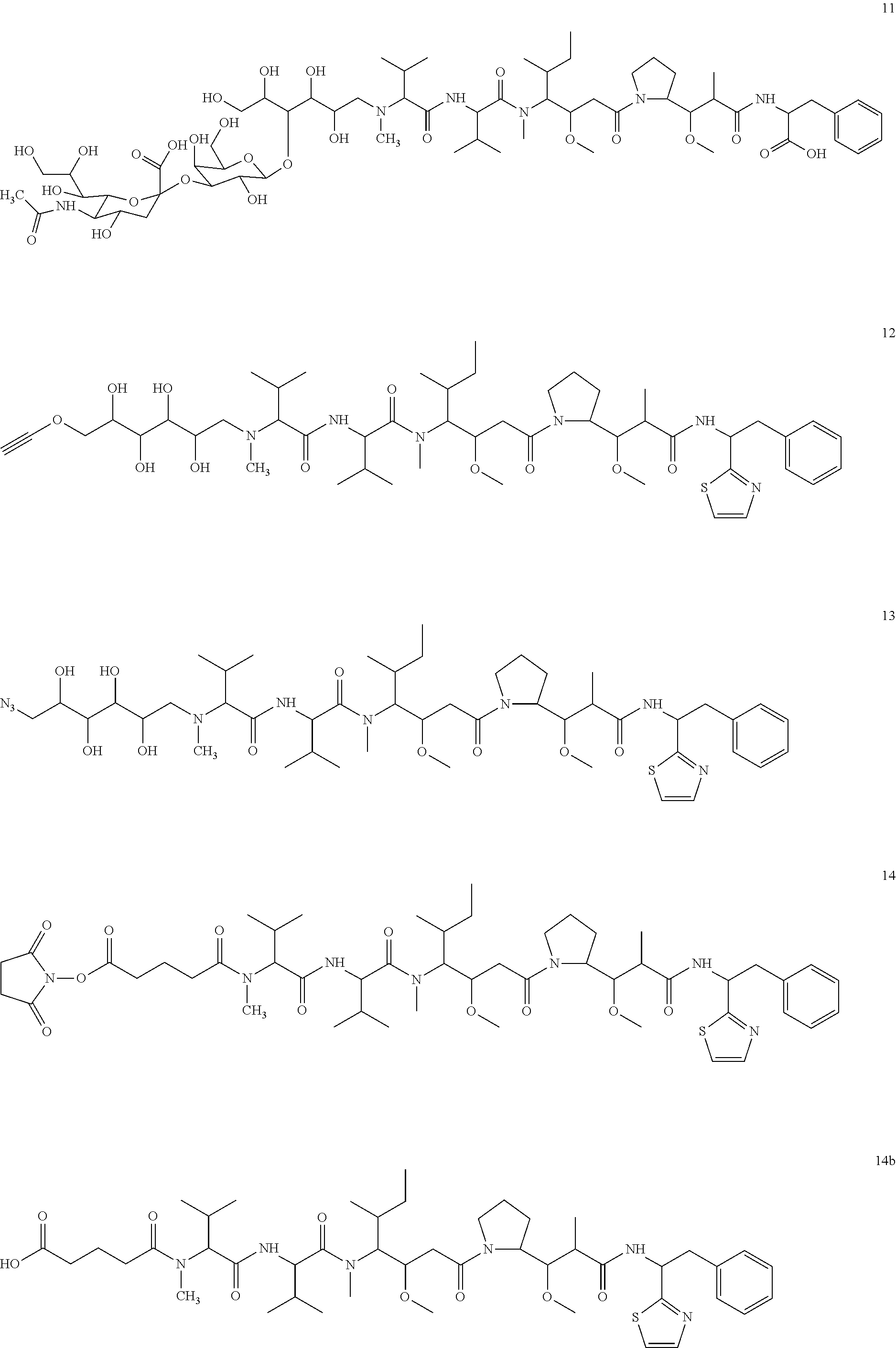

N-(6-O-propargyl-D-galactosyl)-MMAF (3): sodium cyanoborohydride (200 μmol) and 6-O-propargyl-D-galactose (45 μmol) were added to the solution of MMAF (2.7 μmol) in dimethylsulphoxide (0.7 ml). The mixture was stirred at 60° C. for three days.

N-(6-azido-6-deoxy-D-galactosyl)-MMAF (4): sodium cyanoborohydride (160 μmol) and 6-azido-6-deoxy-D-galactose (95 μmol) were added to the solution of MMAF (2.7 μmol) in DMSO (0.6 ml). The mixture was stirred at 60° C. for three days.

N-(2-deoxy-D-glucosyl)-MMAF (5): sodium cyanoborohydride (28 μmol) and 2-deoxy-D-glucose (21 μmol) were added to the solution of MMAF (1.4 μmol) in DMSO (0.6 ml). The mixture was stirred at 60° C. for three days.

N-(3-butynyl)-MMAF (6): to the solution of MMAF (2.7 μmol) in dry DMF (0.6 ml) was added NaH (54 μmol) and 4-bromo-1-butyne (27 μmol). The mixture was stirred at 60° C. for 4 hours. Reaction was quenched by adding dry methanol (0.2 ml).

N-(4-pentynyl)-MMAF (7): to the solution of MMAF (1.4 μmol) in dry DMF (0.4 ml) was added NaH (7 μmol) and 5-iodo-1-pentyne (7 μmol). The mixture was stirred at room temperature for 3 hours. Reaction was quenched by adding dry methanol (0.2 ml).

N-[6-O-(β-D-galactopyranosyl)-D-galactosyl]-MMAF (8): sodium cyanoborohydride (25 μmol) and 6-O-(β-D-galactopyranosyl)-D-galactose (5.3 μmol) were added to the solution of MMAF (0.7 μmol) in DMSO (0.25 ml). The mixture was stirred at 60° C. for five days.

N-[2-acetamido-2-deoxy-4-O-(β-D-galacto-pyranosyl)-D-glucosyl)-MMAF (9): sodium cyanoborohydride (50 μmol) and 2-acetamido-2-deoxy-4-O-(β-D-galactopyranosyl)-D-glucose (11 μmol) were added to the solution of MMAF (1.4 μmol) in DMSO (0.4 ml). The mixture was stirred at 60° C. for five days.

N-{4-O-[4-O-(α-D-galactopyranosyl)-β-D-galactopyranosyl]-D-glucosyl}-MMAF (10): sodium cyanoborohydride (50 μmol) and 4-O-[4-O-(α-D-galactopyranosyl)-β-D-galactopyranosyl]-D-glucose (11 μmol) were added to the solution of MMAF (1.4 μmol) in DMSO (0.4 ml). The mixture was stirred at 60° C. for five days.

N-{4-O-[3-O-(α-N-acetylneuraminyl)-β-D-galactopyranosyl]-D-glucosyl}-MMAF (11): sodium cyanoborohydride (50 μmol) and 4-O-[3-O-(α-N-acetyl-neuraminyl)-β-D-galactopyranosyl]-D-glucose (11 μmol) were added to the solution of MMAF (1.4 μmol) in DMSO (0.4 ml). The mixture was stirred at 60° C. for five days.

N-(6-O-propargyl-D-galactosyl)-dolastatin 10 (12): sodium cyanoborohydride (200 μmol) and 6-O-propargyl-D-galactose (45 μmol) were added to the solution of momomethyl-dolastatin 10 (2.5 μmol) in DMSO (0.7 ml). The mixture was stirred at 60° C. for three days.

N-(6-azido-6-deoxy-D-galactosyl)-dolastatin 10 (13): sodium cyanoborohydride (160 μmol) and 6-azido-6-deoxy-D-galactose (95 μmol) were added to the solution of momomethyl-dolastatin 10 (2.5 μmol) in DMSO (0.6 ml). The mixture was stirred at 60° C. for three days.

N—(N-hydroxysuccinimidylglutaryl)-MMAF (14): disuccinimidyl glutarate (20 μmol) and diisopropylethylamine (20 μmol) were added to the solution of MMAF (1.4 μmol) in ACN (0.4 ml). The mixture was stirred at room temperature overnight. To produce N-glutaryl-MMAF (14b), an aliquot of (14) was hydrolyzed in aqueous solution.

The products were purified by Äkta purifier 10 (GE Healthcare) HPLC instrument with Gemini-NX-5 u C-18 reverse-phase column (4.6×250 mm, 110 Å (Phenomenex)) eluted with ACN gradient in aqueous ammonium hydrogen carbonate or aqueous trifluoroacetic acid.

For example N-(2-deoxy-D-glucosyl)-MMAF (5) eluted with lower ACN concentration at 19.6 min (about 37% ACN) before both the original MMAF (1) at 21.7 min (about 40% ACN) and N-(3-butynyl)-MMAF (6) at 26.0 min (about 45% ACN), showing that it was more hydrophilic.

Matrix-assisted laser desorption-ionization time-of-flight (MALDI-TOF) mass spectra were recorded on a Bruker Ultraflex III TOF/TOF mass spectrometer (Bruker Daltonics, Bremen, Germany) using 2,5-dihydroxybenzoic acid matrix: (3) m/z=956 [M+Na], (4) m/z=943 [M+Na], (5) m/z=902 [M+Na], (6) m/z=806 [M+Na], (7) m/z=820 [M+Na], (8) m/z=1080 [M+Na], (9) m/z=1121 [M+Na], (10) m/z=1242 [M+Na], (11) m/z=1371 [M+Na], (12) m/z=995 [M+Na], (13) m/z=982 [M+Na], (14) m/z=868 for hydrolyzed NHS [M+Na].

### Example 2

### In Vitro Cytotoxicity of Dolastatin Derivatives

Human ovarian cancer cell line SKOV-3 was from the ATCC (Manassas, Va., USA). The cells were grown according to the manufacturer's recommendations. Log phase cultures were collected and 5000 cells/well were seeded onto 96-well plates and incubated for 24 h. Serial dilutions of test molecules from a stock solution of 100 μM in 10% DMSO were made in cell culture medium, added to cells (maximum concentration of dimethylsulphoxide was 1%) and cultures were incubated further for 96 h. Cell viability was evaluated using PrestoBlue cell viability reagent (Life Technologies, Carlsbad, Calif., USA) according to the manufacturer's instructions. Cells were incubated for 2 h, and dye reduction was measured by absorbance at 570 nm. The compounds were assayed 1-2 times in triplicate.

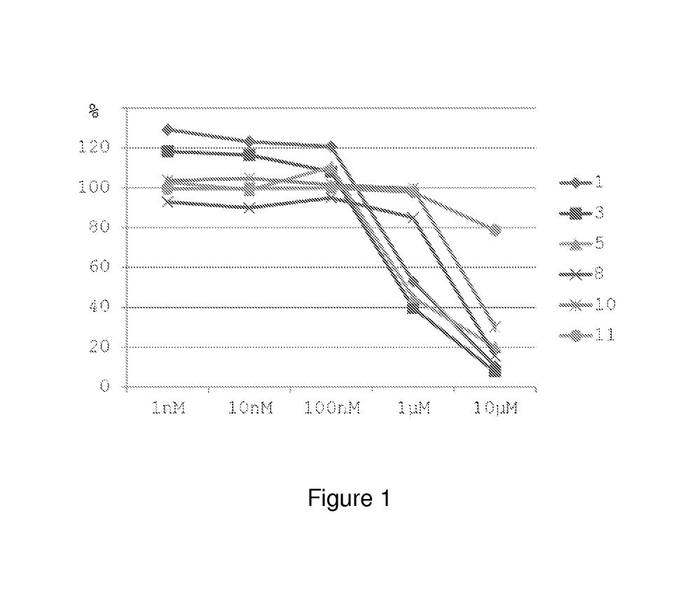

Results of an exemplary assay are shown in FIG. 1, in which compound numbering is according to Example 1. The results are expressed in Table 1 as IC50 values of the analyzed derivatives. In conclusion, 1) all the analyzed alkyl derivatives of MMAF and dolastatin 10 were cytotoxic against SKOV-3 ovarian cancer cells; 2) monosaccharide derivatives 3, 4 and 5 were equally or only slightly less cytotoxic as 1, and monosaccharide derivatives 13 and 14b were equally or only slightly less cytotoxic as 2, showing that the amine conjugates of saccharides and MMAF or monomethyldolastatin 10 have preserved capability to bind to tubulin; 3) oligosaccharide derivatives 8, 11 and 12 were less cytotoxic than 1 when applied to the cell culture medium, reflecting their high hydrophilicity and lowered ability to pass through cellular membranes; and 4) the hydrophobic alkyl derivative 6 was more cytotoxic than 1, showing that a hydrophobic linker increases the ability of the conjugate to pass through cellular membranes.

TABLE 1
Cytotoxicity of dolastatin derivatives.

| Compound | IC50 ¹⁾ | |
|---|---|---|
| 1 | 0.1-1 | μM |
| 14b | 0.1-1 | μM |
| 3 | 0.1-10 | μM |
| 4 | 0.1-1 | μM |
| 5 | 1 | μM |
| 6 | <1 | nM ²⁾ |
| 8 | 1-10 | μM |
| 10 | 1-10 | μM |
| 11 | >10 | μM ²⁾ |
| 2 | <1 | nM ²⁾ |
| 12 | 1 | nM |
| 13 | <1 | nM ²⁾ |

¹⁾ IC50 values were determined as the concentration range wherein SKOV-3 ovarian cancer cell viability falls to 50%.
²⁾ The measured range was between 1 nM-10 μM.

### Example 3

### Synthesis of Peptide-Linker-Payload Molecule Conjugates

5.1 mg (11 μmol) tetrapeptide H₂N-Leu-Ser-Lys-Leu-CONH₂ (Bachem, Bubendorf, Switzerland), capable of binding to thrombospondin-1 (Ribeiro et al., 1999. J. Biol. Chem. 274:13586-93) and thus internalization into e.g. low density lipoprotein receptor-related protein-1 LRP-1 expressing cells (Greenaway et al., 2007. J. Cell Physiol. 210:807-18), was incubated overnight at room temperature with 4.2 mg (11 μmol) NHS-PEG₄-azide (Pierce Biotechnology, Rockford, Ill., USA) in 260 μl dimethylsulphoxide (DMSO) to form a product with m/z 754.4 in MAL-DI-TOF mass spectrometry with α-cyano-4-hydroxycinnaminic acid matrix. A 35 nmol aliquot from the reaction mixture, corresponding to α-amido and ε-amido peptide conjugates, was combined with 35 nmol CuSO₄, 175 nmol sodium ascorbate and 35 nmol N-(6-O-propargyl-D-galactosyl)-MMAF (3) in 30% (v/v) aqueous DMSO and incubated at room temperature for two hours to form mixture of 15a and 15b. The product was detected at m/z 1688.0 (calc. m/z 1688 for [M+Na]+ adduct ion) in MALDI-TOF mass spectrometry.

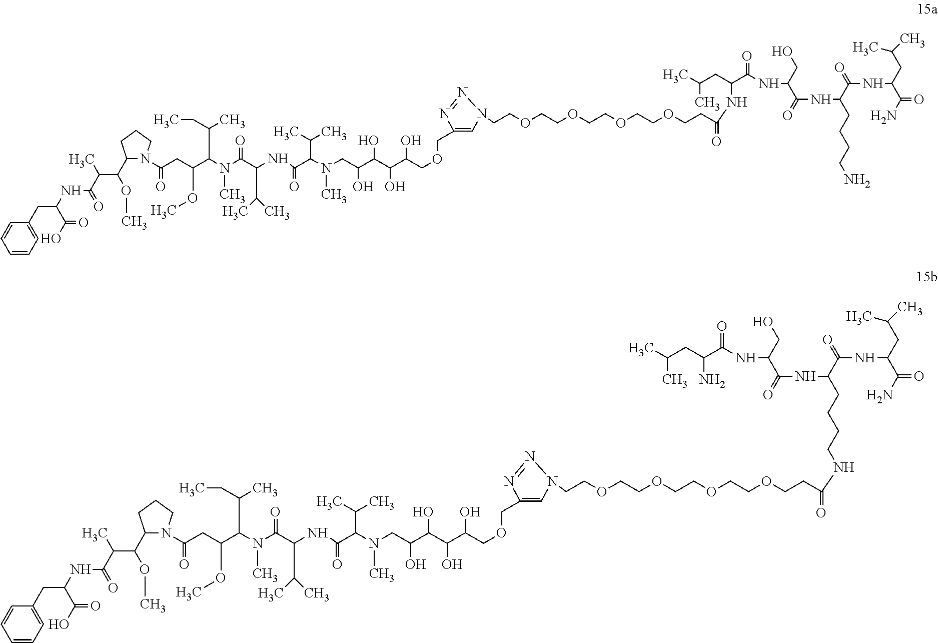 [SMILES: CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NC(CC(C)C)C(=O)NC(CO)C(=O)NC(CCCCN)C(=O)NC(CC(C)C)C(N)=O)nn1)C(C)C ; CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NCCCCC(NC(=O)C(CO)NC(=O)C(N)CC(C)C)C(=O)NC(CC(C)C)C(N)=O)nn1)C(C)C]

### Example 4

### Synthesis of Antibody-Drug Conjugates

Lysine side chains of a monoclonal antibody, e.g. the human-mouse chimeric IgG1 antibody cetuximab (Merck KGaA), the humanized IgG1 antibody trastuzumab (Roche) or the human IgG2 antibody panitumumab (Amgen), are modified with 1) azide by N-hydroxysuccinimide (NHS) ester activated azide, e.g. NHS-PEG4-azide or 2-azidoacetic acid NHS ester, commercially available from e.g. Pierce Biotechnology (Rockford, Ill., USA), or 2) alkyne by NHS ester activated alkyne, e.g. 3-propargyloxy-propanoic acid NHS ester commercially available e.g. from Cambio (Cambridge, UK); in a reaction mixture containing 0.1-10 g/l (e.g. about 5 g/l) antibody and 2-50 fold (e.g. about 10-fold) molar excess of NHS-ester activated azide or alkyne (e.g. about 0.3 mM for 5 g/l antibody solution) in antibody-compatible non-amine aqueous buffer at pH between about 7-8 (e.g. 50 mM sodium phosphate buffer pH 7.3); with incubation at from about 0° C. to about 60° C. (e.g. at room temperature i.e. about 20° C.) for from about 0.5 hour to about 24 hours (e.g. five hours at about 20°). The exact molar excess of the modification reagent, reaction temperature and reaction time are optimized for each antibody individually so that antigen binding and effector functions of the antibody are not compromised, or to about on average 2-4 modified lysine side chains per antibody molecule unless otherwise indicated. After the reaction the modified antibody and non-reacted modification reagent are separated by e.g. purification of the antibody by protein G chromatography, filtration or dialysis, or other well-known methods.

Preparation of the antibody-drug conjugates is done by Cu(I)-catalyzed alkyne-azide cycloaddition of alkyne-linker-MMAF, e.g. (3), in the presence of Cu(I) stabilizing biocompatible chelator, forming a covalent triazole bond to the linker-MMAF conjugate. The azido-modified protein is dissolved in suitable aqueous buffer solution, e.g. 25 mM sodium phosphate, pH 7.3, and per 1 mol of azido-groups in the protein, 2 mol of alkyne-linker-MMAF, 0.5 mol of CuSO₄, 5 mol of sodium ascorbate, 2.5 mol of Cu(I) stabilizing biocompatible chelator (e.g. Tris[(1-benzyl-1H-1,2,3-triazol-4-yl)methyl]amine, TBTA) are added. Reaction is conducted at RT for 2 hours. The conjugated antibody is purified by size exclusion to give a product with on average 1-4 MMAF per antibody molecule.

### Example 5

### Synthesis of TGTA (tris{[1-(6-D-Galactosyl)-1H-1,2,3-triazol-4-yl]methyl}amine)

General experimental details: Reagents and solvents were purchased from commercial sources. Reaction solvents were dried and distilled prior to use when necessary. All reactions containing moisture- or air-sensitive reagents were carried out under an argon atmosphere. The preparation of 1 has been described previously and similar routes were employed in the current synthesis (see for example Yang, J., et al., 2003. J. S. Org. Lett. 5:2223-6).

The NMR spectra were recorded with a Bruker Avance spectrometer operating at 600 MHz (¹H: 600 MHz, ¹³C: 150 MHz). Pulse sequences provided by the manufacturer were utilized. The probe temperature during the experiments was kept at 22° C. unless otherwise mentioned. Chemical shifts are expressed on the δ scale (in ppm) using TMS (tetramethylsilane), residual chloroform, acetone, H₂O or methanol as internal standards. Coupling constants are given in Hz and provided only once when first encountered. Coupling patterns are given as s, singlet, d, doublet, t, triplet etc. Mass spectra were obtained with a Bruker Ultraflex III MALDI-TOF mass spectrometer operated in positive/negative mode. TLC was performed on aluminium sheets precoated with silica gel 60 F254 (Merck). Flash chromatography was carried out on silica gel 60 (0.040-0.060 mm, Aldrich). Spots were visualized by UV followed by charring with 1:5 H₂SO₄/MeOH and heating.

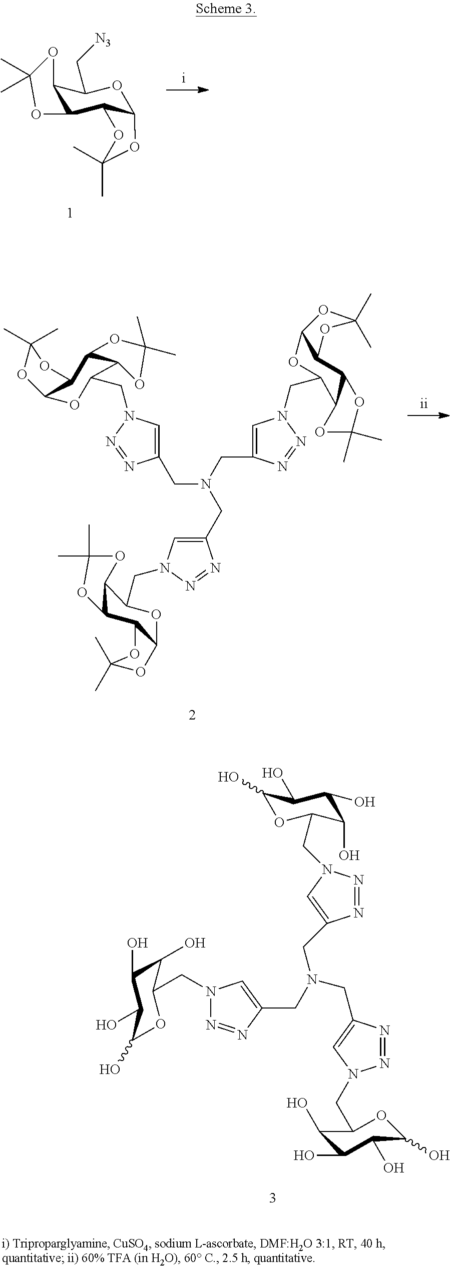

Protected TGTA (2): To a solution containing 43 mg of 1 (0.15 mmol, 5 equiv.) and 4.3 μl tripropargylamine (0.03 mmol, 1 equiv.) in 2 ml of DMF:H₂O (3:1) was added 2.4 mg CuSO₄ (0.015 mmol, 0.5 equiv.) and 6.4 mg sodium L-ascorbate (0.03 mmol, 1 equiv.). The resulting mixture was stirred at RT for 40 h (during this time a white solid precipitated from the reaction mixture). After 40 h, the reaction mixture was diluted with 20 ml EtOAc transferred to a separatory funnel and washed with 5 ml NH₄Cl-solution (prepared by dissolving a saturated NH₄Cl-solution with equal amount of water 1:1 v/v) and 15 ml brine. The organic phase was dried with Na₂SO₄, filtered and concentrated to give the crude product. The crude product was purified by column chromatography (EtOAc-EtOAc:MeOH 3:1) to give 2 as a colorless oil (30 mg, quantitative). TLC: R_{f}=0.22 (EtOAc). ¹H NMR (600 MHz, CDCl₃, 25° C.): δ=8.56 (s, 3H, triazole-H), 5.48 (d, 3H, J_{1,2}=5.0 Hz, H-1), 4.67 (dd, 3H, J_{6a,5}=3.1, J_{6a,6b}=14.1 Hz, H-6a), 4.65 (dd, 3H, J_{3,2}=2.5, J_{3,4}=8.1, H-3), 4.58 (dd, 3H, J_{6b,5}=9.0 Hz, H-6b), 4.41 and 4.33 (each d, each 3H. J_{NCH2a,NCH2b}=14.1 Hz, N(CH₂)₃), 4.32 (dd, 3H, H-2), 4.25 (dd, 3H, J_{4,5}=1.4 Hz, H-4), 4.17 (ddd, 3H, H-5), 1.50, 1.39, 1.37 and 1.25 (each s, each 9H, O₂C(CH₃)₂) ppm. HRMS: calcd. for C₄₅H₆₆N₁₀O₁₅Na [M+Na]⁺ 1009.46. found 1009.40.

TGTA (3): 33 mg of 2 (0.034 mmol) was dissolved in 3 ml 60% TFA (in H₂O) and stirred at 50° C. for 1.5 hours. The reaction mixture was then diluted with water, concentrated and dried under vacuum to give 3 as a white solid (25 mg, quantitative, α:β 2:3). Selected NMR-data; ¹H NMR (600 MHz, D₂O, 25° C.): δ=8.32 (s, 6H (α and β, 3H each), triazole-H), 5.21 (d, 3H, J_{1,2}=3.9 Hz, H-1α), 4.59 (s, 12H (α and β, 6H each), N(CH₂)₃), 4.50 (d, 3H, J_{1,2}=8.1 Hz, H-1β). HRMS: calcd. for C₂₇H₄₂N₁₀Na [M+Na]⁺ 769.27. found 769.23.

The structure of TGTA and its proposed copper(I) chelating mode:

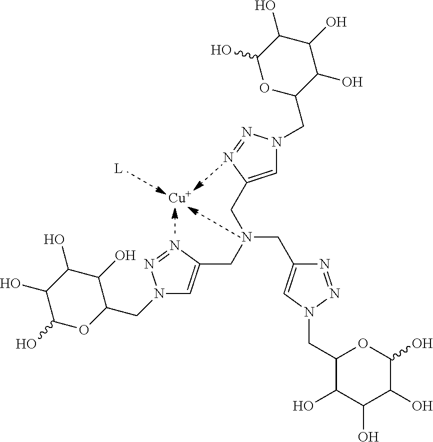

### Example 6

### Synthesis of Cetuximab and Omalizumab Drug Conjugates by Copper(I) Catalyzed Click Reaction

To introduce azide-groups into cetuximab or omalizumab, 100 μg (670 pmol) of antibodies in 25 mM sodium phosphate buffer pH 7.3 (80 μl) was incubated with 15-20 molar excess of NHS-PEG₄-N₃ in DMSO (2 μl) for 2 hours at room temperature. Low molecular weight reagents were removed by Amicon centrifugal filter unit, 30K, with repeated addition of PBS.

Antibody-drug conjugates were generated by azide-alkyne cycloaddition reaction as follows: To cetuximab-PEG₄-N₃ or omalizumab-PEG₄-N₃ in 25 mM sodium phosphate buffer pH 7.3 (40 μl) was added 20-40× molar excess of N-(6-propargyl-D-galactosyl)-dolastatin 10 in DMSO (2 μl), followed by 10× molar excess of CuSO₄ in H₂O (1 μl), 50× molar excess of sodium ascorbate in H₂O (1 μl) and 25× molar excess of TGTA in H₂O (1 μl). Reactions were allowed to proceed at room temperature for 0.5 hour. The drug conjugated antibodies were purified by Amicon centrifugal filter units, 30K, with repeated addition of PBS.

The drug-antibody-ratio (DAR) in the conjugates was calculated by isolating the Fc-fragments and light chains. Fc-fragments were released by FabRICATOR enzyme (34 U) at 37° C. for 1.5 hours and recovered with self-manufactured Poros R1 tips by elution with 60% ACN in 0.1% TFA (5 μl). The remaining F(ab′)2 in Poros R1 tip was denatured with 6M guanidine-HCl (30 μl) at 60° C. for 0.5 hour. Disulfide bonds were reduced with 0.1 M dithiothreitol (20 μl) at 60° C. for 0.5 hour. Light chains were eluted with 60% ACN, 0.1% TFA (5 μl). Fc-fragments and light chains were analysed by MALDI-TOF MS using either sinapinic acid or 2,5-dihydroxyacetophenone as the matrix.

### Example 7

### Synthesis of 2G12-Drug Conjugates by Copper(I) Catalyzed Click Reaction

To 300 μg (2 nmol) of 2G12 antibody (human IgG; Polymun Scientific, Austria) in PBS—5% mannitol—0.1% Tween20 solution (80 μl) was added 10× molar excess of NHS-PEG₄-N₃ dissolved in DMSO (1 or 4 μl) and the reaction was allowed to proceed for 3 hours at room temperature. To minimize loss of antibody, the reaction was conducted in an Amicon Ultra centrifugal filter unit (30K, 0.5 ml). Low molecular weight reagents were then removed by Amicon centrifugal filter with repeated addition of PBS—5% mannitol—0.1% Tween20.

To prepare 2G12-drug conjugate, 50 molar excess of TGTA and sodium ascorbate in H₂O (2 μl), 10 molar excess of CuSO₄ in H2O (0.34 μl, final concentration 500 μM) and 20 molar excess of N-(6-propargyl-D-galactosyl)-dolastatin 10 in DMSO (4 μl) were added into the solution of 2 nmol of 2G12-PEG₄-N₃ (40 μl). The reactions were performed at room temperature for 45 minutes. The conjugated antibodies were purified by Amicon centrifugal filter as above.

The DAR of the 2G12-drug conjugate was calculated by isolating the Fc-fragments and light chains followed by MALDI-TOF MS analysis as described in Example 6.

### Example 8

### Synthesis of Trastuzumab-Drug Conjugates by Copper(I) Catalyzed Click Reaction

To introduce azide-groups into trastuzumab, 10 mg of antibody in 25 mM sodium phosphate buffer pH 7.3 (80 μl) was incubated with (1) 10× molar or (2) 50× molar excess of NHS-PEG₄-N₃ in DMSO (1.8 μl and 8.8 μl) for 2 hours at room temperature. Low molecular weight reagents were removed by Amicon centrifugal filter unit, 30K, with repeated additions of PBS.

Antibody-drug conjugates were generated by azide-alkyne cycloaddition reaction as follows: To 8 mg of trastuzumab-PEG₄-N₃ samples in 25 mM sodium phosphate buffer pH 7.3 (1153 μl) was added 20× molar excess of N-(6-propargyl-D-galactosyl)-dolastatin 10 in DMSO (10 μl), followed by 10× molar excess of CuSO₄ in H₂O (2 μl), 50× molar excess of sodium ascorbate in H₂O (5 μl) and 50× molar excess of TGTA in H₂O (30 μl). Final reaction volumes were 1200 μl. Reactions were allowed to proceed at room temperature for 1 hour. The drug conjugated antibodies were purified by Amicon centrifugal filter units, 30K, with repeated additions of PBS.

The DAR of the trastuzumab-drug conjugates was calculated by isolating the Fc-fragments and light chains followed by MALDI-TOF MS analysis as described in Example 6. The DAR obtained with reaction (1) was on average 1, while that with reaction (2) was about 2-3.

### Example 9

### Conjugation of Monomethyldolastatin (MODO) by Val-Cit-PAB Linker to Cetuximab

### Val-Cit-PAB-MODO

6.5 mg (8 μmol) MODO in DMF (200 μl), 2 molar excess of Fmoc-Val-Cit-PAB-pnp, 0.3 mg (2 μmol) HoBt in DMF (28 μl), 7 μl (40 μmol) DIPEA and 65 μl DMF were stirred for two days at room temperature. The crude reaction mixture was analysed by MALDI-TOF mass spectra using 2,5-dihydroxybenzoic acid matrix, showing expected mass for Fmoc-Val-Cit-PAB-MODO (m/z 1420 [M+Na]).

Fmoc was removed by adding 150 μl of diethylamine and by stirring at room temperature overnight. MALDI-TOF mass analysis using 2,5-dihydroxybenzoic acid matrix showed the generation of expected deprotected product (m/z 1198 [M+Na]).

Val-Cit-PAB-MODO was purified by Äkta purifier (GE Healthcare) HPLC instrument with Gemini 5 μm NX-C18 reverse phase column (21.1×250 mm, 110 Å, ÄXIA (Phenomenex)) eluted with ACN gradient in aqueous ammonium acetate.

### Alkyne-Val-Cit-PAB-MODO

15 mg (67 μmol) of 3-propargyloxypropionic acide NHS-ester (Cambio, Dry Drayton, Cambs, UK) and 2 mg (24 μmol) sodium hydrogen carbonate were added to the solution of Val-Cit-PAB-MODO (6.4 μmol) in 75% DMSO (1 ml). The mixture was stirred at room temperature for two days. The product was analysed by MALDI-TOF MS, showing the expected product (m/z 1308 [M+Na]).

Alkyne-Val-Cit-PAB-MODO was purified by Äkta purifier (GE Healthcare) HPLC instrument with Gemini 5 μm NX-C18 reverse phase column (4.6×250 mm, 110 Å (Phenomenex)) eluted with ACN gradient in aqueous ammonium acetate.

### PEG-N₃-Cetuximab

1 mg (6.7 nmol) of cetuximab in PBS (150 μl) was incubated with 10 molar excess of N₃-PEG-NHS (Pierce) in DMSO (9 μl) for 2 hours at room temperature. Non-reacted N₃-PEG-NHS was separated by Amicon centrifugal filter unit, 30K.

To verify the PEG-azide attachment, antibody light chains were released by denaturating the antibodies with 6M guanidine-HCl at 60° C. for 0.5 hours, followed by disulfide reduction with 0.1 M dithiothreitol at 60° C. for 0.5 hour. Light chains were purified from reaction mixture with self-manufactured miniaturized Poros R1 columns by eluting them with 60% ACN in 0.1% TFA (5 μl). Light chain analysis was performed by MALDI-TOF MS, which confirmed the presence of PEG-azide units (+273 Da).

Val-Cit-PAB-MODO-Cetuximab (cetuximab-VC-MODO)

The title drug-antibody conjugate (Scheme 4) was generated by a copper(I) catalyzed click reaction containing 3.2 nmol PEG-N₃-Cetuximab in PBS (90 μl), 32 nmol Alkyne-Val-Cit-PAB-MODO in DMSO (125 μl), 1250 nmol TGTA in MQ (90 μl), 1250 nmol Na-ascorbate in MQ (12.6 μl), 250 nmol of CuSO₄ in MQ (5 μl) and PBS (reaction volume 0.5 ml). The mixture was allowed to react for 1 hour at RT. Antibody conjugate was purified in Amicon centrifugal filter unit, 30K.

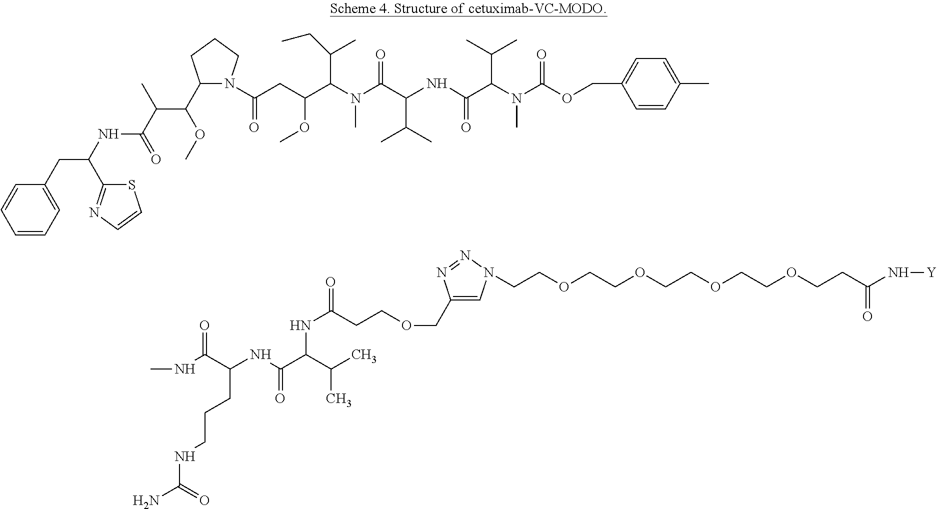

To calculate the drug-antibody-ratio (DAR), cetuximab-VC-MODO was subjected to Fc-fragment and light chain isolation. Fc-fragments were released by FabRICATOR enzyme (Genovis AB, Lund, Sweden) overnight at 37° C. and purified with Poros R1 tips. Fc-fragments were eluted with 60% ACN, 0.1% TFA (5 μl). Light chains were released by 6M guanidine-HCl and dithiothreitol as above, and recovered using Poros R1 tips. Based on MALDI-TOF MS analysis of these protein domains, the drug-antibody-ratio was on average 1.5.

### Example 10

### Synthesis of Epothilone-Linker Conjugates N-(propargylgalactose)aminoepothilone

2 mg of 21-aminoepothilone B (3.8 μmol) was dissolved in 200 μl DMSO. 200 μmol of sodium cyanoborohydride in DMSO (200 μl) and 76 μmol of 6-propargylgalactose in DMSO (100 μl) were added. Reaction was allowed to proceed overnight at 60° C. MALDI-TOF MS analysis showed the formation of N-(6-propargylgalactosyl)aminoepothilone, m/z=747 [M+Na]⁺. Sample 5 was purified by HPLC on Gemini-NX-5 u C-18 reverse-phase column eluted with acetonitrile gradient in ammonium acetate buffer pH 5.6.

N-(2-deoxyglucosyl)aminoepothilone

2 mg of 21-aminoepothilone B (3.8 μmol) was dissolved in 200 μl DMSO. 500 μmol of sodium cyanoborohydride in DMSO (500 μl) and 380 μmol of 2-deoxyglucose in DMSO (200 μl) were added. Reaction was allowed to proceed overnight at 60° C. MALDI-TOF MS analysis showed the formation of N-(2-deoxyglucosyl)aminoepothilone, m/z=693 [M+Na]. Sample was purified by HPLC on Gemini-NX-5 u C-18 reverse-phase column eluted with acetonitrile gradient in ammonium acetate buffer pH 5.6.

### Example 11

### In Vitro Cytotoxicity of Drug Derivatives, Cell Binder-Drug Conjugates and Antibody-Drug Conjugates

Human ovarian cancer cell line SKOV-3 (EGFR⁺HER2⁺), head-and-neck squamous cell carcinoma cell line HSC-2 (EGFR⁺) and multidrug-resistant colorectal carcinoma cell line LS513 (EGFR⁺) were from the ATCC (Manassas, Va., USA). The cells were grown according to the manufacturer's recommendations. In vitro cytotoxicity assays with the cells were performed as in Example 2 above.

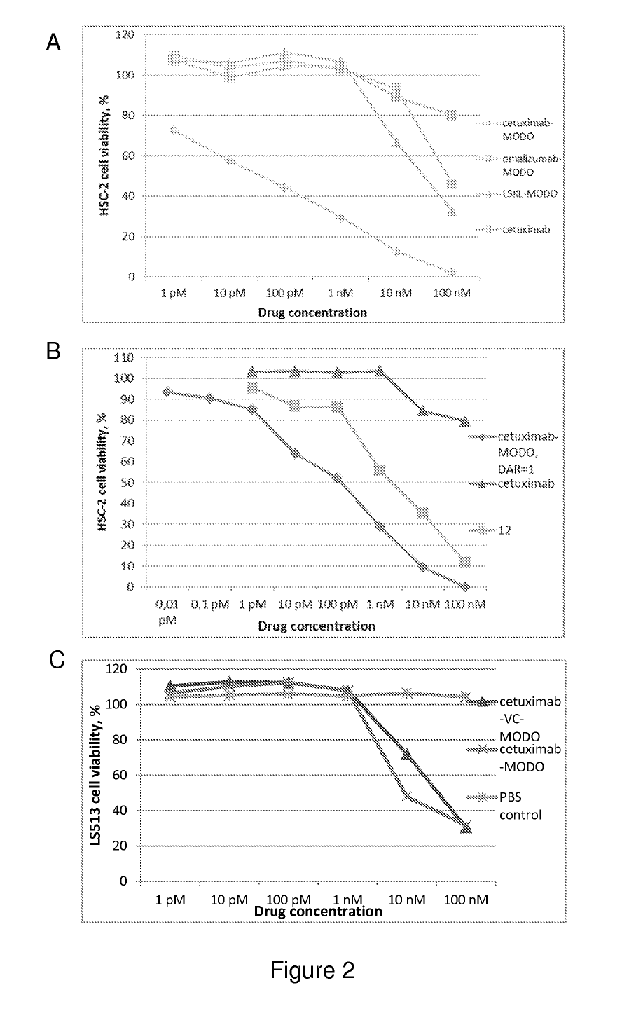

Results of exemplary assays are shown in FIG. 2, in which cetuximab-MODO and omalizumab-MODO (monomethyldolastatin 10) conjugates were prepared by copper(I) catalyzed click reaction between compound 12 and azide-modified antibodies. Cetuximab-VC-MODO contains valine-citrulline peptidase sensitive linker without a hydrophilic linker moiety. LSKL-MODO was prepared by conjugating the tetrapeptide with MODO as described in the Examples. All drug concentrations in the y-axis were normalized to actual monomethyldolastatin 10 drug content in each conjugate. A, Cytotoxicity assay with HSC-2 head-and-neck cancer cells. Both cetuximab-MODO and LSKL-MODO were more effective against the cancer cells than either the unspecific antibody-drug conjugate prepared from omalizumab or unconjugated cetuximab. B, Cytotoxicity assay with HSC-2 head-and-neck cancer cells. Cetuximab-MODO was more effective against the cancer cells than the unconjugated linker-dolastatin compound 12. C. Cytotoxicity assay with LS513 multi-drug resistant colorectal cancer cells. Cetuximab-MODO (containing hydrophilic linker moiety) was more effective against the drug-resistant cancer cells than cetuximab-VC-MODO (containing linker that releases free unconjugated drug inside cells). Results are further expressed in Table 2 as IC50 values of various experiments.

TABLE 2
Cytotoxicity of drug derivatives
and anti-body-drug conjugates.

| Compound | IC50 ¹⁾ |
|---|---|
| cetuximab-MODO | SKOV-3 and HSC-2: |
| | 100 pM-1 nM |
| 2G12-MODO | SKOV-3: 100 pM-1 nM |
| trastuzumab-MODO | SKOV-3: 10 pM-1 nM |
| trastuzumab, cetuxi- | SSKOV-3 and HSC- |
| mab, 2G12 | 2: >100 nM |

¹⁾ IC50 values were determined as the concentration range wherein cancer cell viability falls to 50%.
²⁾ The measured range was between 0.01 pM-10 μM.

In conclusion, 1) antibody-drug conjugates showed increased efficacy against antigen-presenting cells and significantly reduced toxicity against antigen-negative cells; 2) efficient cytotoxicity was demonstrated for cetuximab (mouse-human chimeric IgG1), trastuzumab (humanized IgG1) and 2G12 (human IgG1) antibody-drug conjugates, LSKL tetrapeptide-drug conjugate and unconjugated linker-drug conjugates; and 3) drug with hydrophilic linker was more effective against drug-resistant cells than the same drug without the linker.

### Example 12

### In Vivo Efficacy of Anti-HER2 Antibody-Drug Conjugates

A non-randomized study of anti-HER2 humanized IgG1 antibody-drug conjugates (ADCs; test substances prepared by conjugating N-(6-propargyl-D-galactosyl)-dolastatin 10 to NHS-PEG₄-N₃-modified CHO cell produced trastuzumab as described in the preceding Examples) with two different drug-antibody molar ratios (DAR=1 and DAR=3), unconjugated anti-HER2 humanized IgG1 (trastuzumab) and control (phosphate buffered saline, PBS) was carried out in a xenograft nude mouse model to evaluate in vivo efficacy of the ADCs. The study was conducted according to standard guidelines of the test facility and was approved by appropriate ethical committee (University of Turku and Turku University Hospital, Turku, Finland).

Human cancer cell line SKOV-3 (ovarian adenocarcinoma) was implanted s.c. (3×10⁶ cells in 50% matrigel) in one flank of female, adult Harlan HSD:athymic nude Foxn1^{nu} mice. The first dose of the test or control substances was administered when the tumors had grown to average volume of 100 mm³ (4-8 mm diameter). Tumor length (L) and width (W) were recorded in mm. Tumor volumes (V) in mm³ were calculated according to the formula V=½L×W². Mice with different sized tumors were equally divided into study groups to obtain homogenous groups.

TABLE 3
Study groups and dosing of test substances.

| Study | Animal | Test | |
|---|---|---|---|
| group | # | substance | Dose |
| I | 1-3 | anti-HER2 ADC, | 10 mg/kg (c. 200 |
| | | DAR = 1 | μg/mouse in 100 μl |
| | | | PBS) |
| II | 4-6 | anti-HER2 ADC, | 10 mg/kg (c. 200 |
| | | DAR = 3 | μg/mouse in 100 μl |
| | | | PBS) |
| III | 7-9 | anti-HER2 IgG1 | 10 mg/kg (c. 200 |
| | | | μg/mouse in 100 μl |
| | | | PBS) |
| IV | 10-12 | PBS | 100 μl PBS |

Test and control substances (Table 3) were administered i.v. three times at seven days' intervals. Tumor volume, animal weight and clinical signs and general behavior of the animals were followed twice weekly. Any unusual signs or behavior were recorded. End-point of the study was when the tumors had reached the maximum allowed diameter (17 mm) or eight weeks after first dosing.

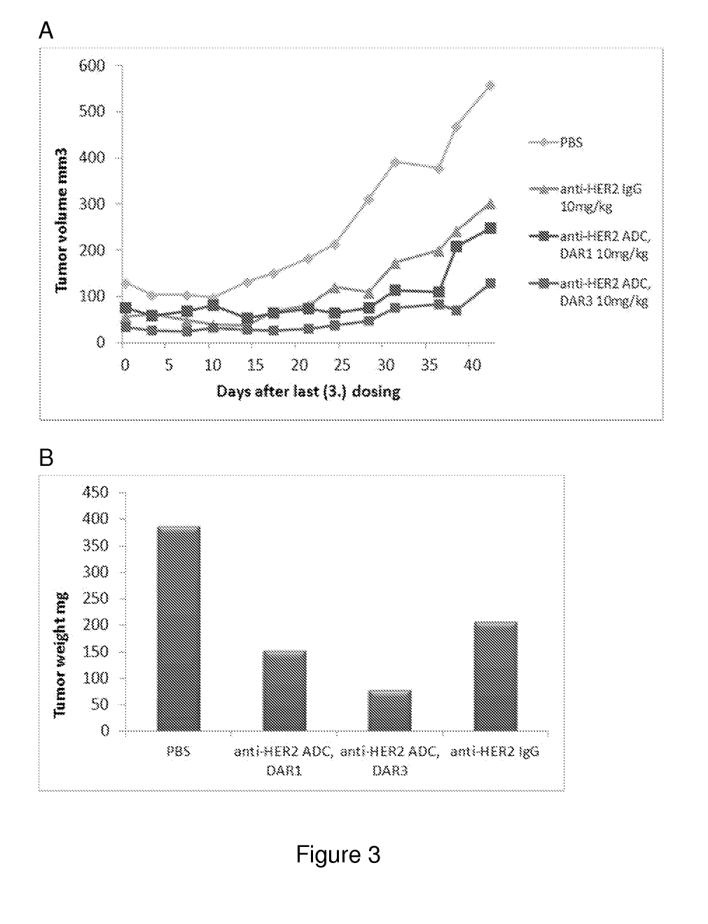

All animals survived for eight weeks (tumor diameter <17 mm) after first dosing of the test or control substances. Anti-HER2 ADC with drug-to-antibody ratio of 3 (DAR=3) showed the highest anti-tumor activity among the tested compounds (FIG. 3A). Both anti-HER2 ADCs with DAR=1 and DAR=3 inhibited tumor growth more than the unconjugated anti-HER2 antibody and control treatment. Average tumor weight in the end of the experiment was less than 20% in anti-HER2 ADC DAR=3 treated mice compared to the control mice receiving only PBS (FIG. 3B). No symptoms of toxicity were observed in the mice during the study.

FIG. 3A, Tumor volume in subcutaneous SKOV-3 xenograft mice treated with anti-HER2 antibody-drug conjugate (ADC; drug-to-antibody ratio DAR=1 or DAR=3), anti-HER2 antibody or PBS (control). FIG. 3B, Average tumor weight in subcutaneous SKOV-3 xenograft mice treated with anti-HER2 ADC (DAR=1 or DAR=3), anti-HER2 antibody or PBS. Tumor weight was recorded eight weeks after the first injection of test or control substances.

### Example 13

### In Vivo Efficacy of Anti-EGFR Antibody-Drug Conjugates

A non-randomized study of the anti-EGFR1 ADC (test substance prepared by conjugating N-(6-propargyl-D-galactosyl)-dolastatin 10 to NHS-PEG₄-N₃-modified CHO cell produced cetuximab as described in the preceding Examples), unconjugated anti-EGFR1 IgG1 and the control substance (phosphate buffered saline) was carried out in a xenograft nude mouse model to evaluate in vivo efficacy of the ADCs towards SKOV-3 s.c. tumors as described in the preceding Example. Test and control substances were administered i.v. three times at seven days' intervals (Table 4).

TABLE 4
Study groups and dosing of test substances.

| Study | Animal | Test | |
|---|---|---|---|
| group | # | substance | Dose |
| I | 1-3 | anti-EGFR1 ADC | 10 mg/kg (c. 200 |
| | | | μg/mouse in 100 μl PBS) |
| II | 4-6 | anti-EGFR1 | 10 mg/kg (c. 200 |
| | | IgG1 | μg/mouse in 100 μl PBS) |
| III | 7-9 | PBS | 100 μl PBS |

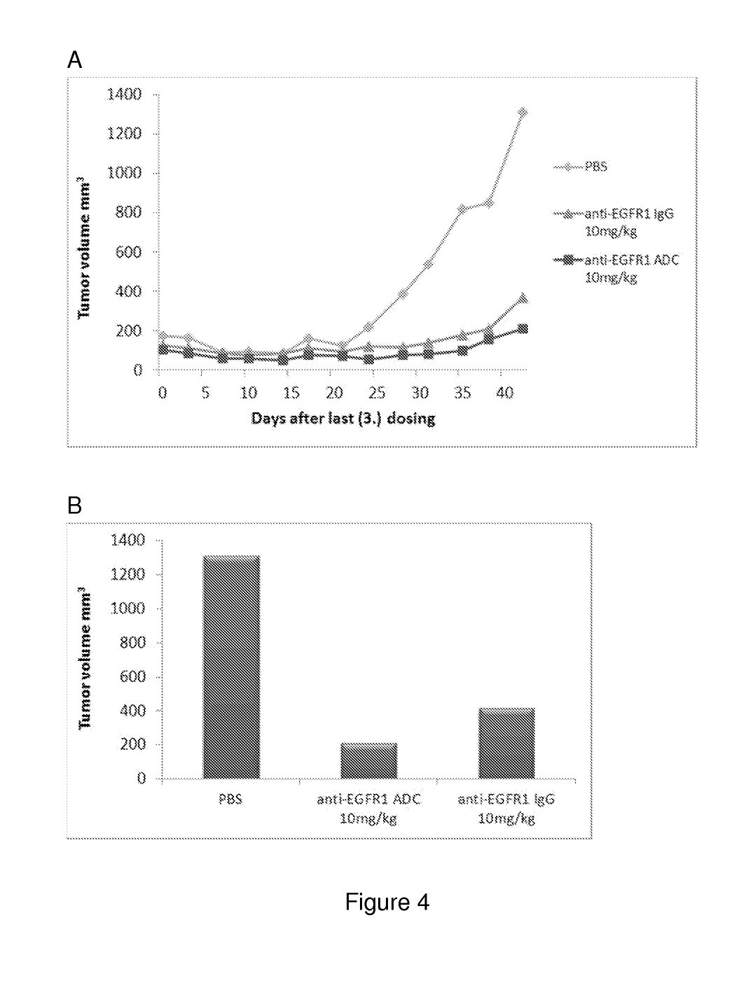

All animals in study groups I and II survived for eight weeks (tumor diameter <17 mm) and were euthanized at the same time. One mouse in the control group (PBS) was euthanized due to a large tumor (diameter exceeding 17 mm) before eight weeks had passed from the dosing. Smallest tumors were observed in the study group I receiving anti-EGFR1 ADC treatment (FIG. 4), showing that the anti-EGFR1 ADC showed anti-tumor activity in vivo in subcutaneous SKOV-3 xenograft nude mice. No symptoms of toxicity were observed in the mice during the study.

FIG. 4A, Tumor volume in subcutaneous SKOV-3 xenograft mice treated with anti-EGFR1 ADC, anti-EGFR1 IgG antibody or PBS (control). FIG. 4B, Average tumor volume in subcutaneous SKOV-3 xenograft mice treated with anti-EGFR1 ADC, anti-EGFR1 IgG antibody or PBS. Tumor volume was recorded eight weeks after the first dosing of test or control substances.

### Example 14

### In Vivo Safety and Clearance Rate of Anti-EGFR1 Antibody-Drug Conjugates

Three anti-EGFR1 ADC doses containing between 5 μg drug/kg and 100 μg drug/kg dolastatin 10 derivative in form of anti-EGFR ADC (test substance prepared by conjugating N-(6-propargyl-D-galactosyl)-dolastatin 10 to NHS-PEG₄-N₃-modified CHO cell produced cetuximab as described in the preceding Examples) were administered to healthy nude mice to evaluate single-dose safety and clearance rate of the test compounds. The study was conducted according to the standard guidelines of the test facility and was approved by appropriate ethical committee (University of Turku and Turku University Hospital, Turku, Finland). Female, adult healthy Harlan HSD:athymic nude Foxn1^{nu} mice were used in the experiment. The test substances were administered i.v. in 100 μl of phosphate buffered saline (Table 5). Serum samples were collected for 13 days after the injection, after which all mice were euthanized.

TABLE 5
Study groups and dosing of test substances.

| | | Test substance | |
|---|---|---|---|
| Study | Animal | (antibody-drug | Amount |
| group | # | conjugate) | (in 100 μl PBS) |
| I | 1-3 | Anti-EGFR1 ADC | 0.5 mg/kg ADC (c. |
| | | | 10 μg/mouse) corre- |
| | | | sponding to c. 5 μg |
| | | | drug/kg |
| II | 4-6 | Anti-EGFR1 ADC | 2.5 mg/kg ADC (c. |
| | | | 50 μg/mouse) corre- |
| | | | sponding to c. 25 μg |
| | | | drug/kg |
| III | 7-9 | Anti-EGFR1 ADC | 10 mg/kg ADC (c. |
| | | | 200 μg/mouse) corre- |
| | | | sponding to c. 100 μg |
| | | | drug/kg |

Human IgG concentrations were measured in the serum samples using anti-human IgG quantification kit (R&D Biotech) and the clearance t^{1/2} was calculated in the 3 phase: t^{1/2} was 5.4 days in group I, 5.1 days in group II, and 4.9 days in group III. The result was similar to t^{1/2} of non-conjugated cetuximab, showing that the ADC had similar clearance rate in healthy mice than the parent antibody.

As is clear for a person skilled in the art, the invention is not limited to the examples and embodiments described above, but the embodiments can freely vary within the scope of the claims.

1. A linker-payload molecule conjugate represented by formula I 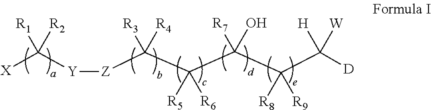
wherein
X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;
Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;
Z is a saccharide or absent;
D is a payload molecule comprising an amine moiety, through which the payload molecule is bound so as to form a secondary or tertiary amine;
R₁, R₂, R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;
W is H, CH₂OH, CH₃, carboxyl, substituted carboxyl, C₁-C₆ alkyl or substituted C₁-C₆ alkyl;
a is an integer from 0 to 6;
b is 0 or 1;
c and e are each independently an integer from 0 to 7; and
d is an integer from 1 to 7.

2. The linker-payload molecule conjugate according to claim 1 represented by formula II 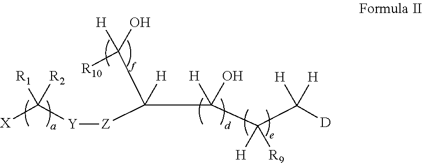
wherein
X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;
Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;
Z is a saccharide or absent;
D is a payload molecule comprising an amine moiety, through which the payload molecule is bound so as to form a secondary or tertiary amine;
R₁, R₂, R₉ and R₁₀ are each independently H, hydroxyl, amine, C₂-C₆ acylamide, carboxyl, substituted carboxyl, C₁-C₆ alkyl or substituted C₁-C₆ alkyl;
a is an integer from 0 to 6;
e is an integer from 0 to 3; and
d and f are integers from 0 to 4 with the proviso that their sum is from 1 to 4.

3. The linker-payload molecule conjugate according to claim 1 represented by formula II 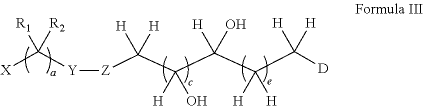
wherein
X is F-E, wherein F is a functional group that can react with an amine, thiol, azide, alkene, alkyne, aldehyde, ketone, carboxylic acid or hydroxylamine in a cell binder, and E is either absent or a polyethyleneoxy unit of formula (CH₂CH₂O)ₚ, wherein p is an integer from 2 to about 20;
Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;
Z is a saccharide or absent;
D is a payload molecule comprising an amine moiety, through which the payload molecule is bound so as to form a secondary or tertiary amine;
R₁ and R₂ are each independently H, hydroxyl, amine, C₂-C₆ acylamide, carboxyl, substituted carboxyl, C₁-C₆ alkyl or substituted C₁-C₆ alkyl;
a is an integer from 0 to 6; and
c and e are each independently an integer from 0 to 3.

4. A cell binder-linker-payload molecule conjugate represented by formula IV 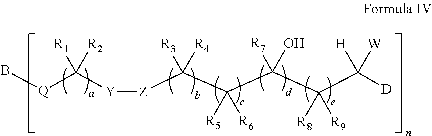
wherein
Y is an oxygen, sulphur, amine, amide, peptide or absent, wherein the peptide is an E₁-P-E₂ unit in which E₁ and E₂ are independently either C═O, O or NRₚ, wherein Rₚ is H, C₁-C₆ alkyl or substituted C₁-C₆ alkyl, P is a peptide unit from 2 to 5 amino acids in length, and E₁ and E₂ can independently be linked to the peptide through the terminal nitrogen, terminal carbon or through a side chain of one of the amino acids of the peptide;
Z is a saccharide or absent;
D is a payload molecule comprising an amine moiety, through which the payload molecule is bound so as to form a secondary or tertiary amine;
R₁, R₂, R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;
W is H, CH₂OH, CH₃, carboxyl, substituted carboxyl, C₁-C₆ alkyl or substituted C₁-C₆ alkyl;
a is an integer from 0 to 6;
b is 0 or 1;
c and e are each independently an integer from 0 to 7;
d is an integer from 1 to 7;
B is a cell binder;
Q is E′-F′-E, wherein F′ is an amine, amide, disulfide, thioether, thioester, hydrazone, Schiff base, oxime, olefin metathesis reaction product, triazole or phosphine group, or other group generated by the reaction of the cell binder with X as defined for formula I, and E and E′ are each independently either absent or a polyethyleneoxy unit of formula (CH₂CH₂O)ₚ, wherein p is an integer from 2 to about 20; and
n is an integer from 1 to about 20.

5. A linker-payload molecule conjugate represented by formula V 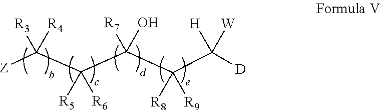
wherein Z is H, OH or a saccharide;
D is a payload molecule comprising an amine moiety, through which the payload molecule is bound so as to form a secondary or tertiary amine;
R₃, R₄, R₅, R₆, R₇, R₈ and R₉ are each independently H, hydroxyl, amine, C₂-C₆ acylamide, carboxyl, substituted carboxyl, C₁-C₆ alkyl or substituted C₁-C₆ alkyl;
W is H, CH₂OH, CH₃, carboxyl, substituted carboxyl, C₁-C₆ alkyl or substituted C₁-C₆ alkyl;
b is 0 or 1;
c and e are each independently an integer from 0 to 7; and
d is an integer from 1 to 7.

6. (canceled)

7. The linker-payload molecule conjugate according to claim 1, wherein D is a cytotoxic agent or a labelling molecule, such as a fluorescent label or a radioactive label.

8. (canceled)

9. The linker-payload molecule conjugate according to claim 1, wherein D is a dolastatin, dolastatin 10, dolastatin 15, auristatin, auristatin F, doxorubicin, epothilone, aminoepothilone or any analogue or derivative thereof.

10. (canceled)

11. (canceled)

12. The linker-payload molecule conjugate according to claim 1, wherein d is 3, 4 or 5; and/or d is 4 and R₇ is H; and/or b is 1 and R₃ and R₄ are each H; and/or a is 1 and R₁ and R₂ are each H; and/or e is 1 and R₈ and R₉ are each H; and/or a, b, c and/or e is 0; and/or W is H; and/or a is 2 or 3 and R₁ and R₂ are both H; and/or Y is an oxygen.

13. (canceled)

14. (canceled)

15. (canceled)

16. (canceled)

17. (canceled)

18. (canceled)

19. (canceled)

20. (canceled)

21. The linker-payload molecule conjugate according to claim 1, wherein X is an amine reacting group, a thiol reactive group, an azide reactive group, an azide, an alkyne reactive group, an alkyne, such as CH≡C, a carbonyl reactive group, or a hydroxylamine reactive group.

22. (canceled)

23. The linker-payload molecule conjugate according to claim 1, wherein the linker-payload molecule conjugate is
N-(6-propargyl-D-galactosyl)-monomethylauristatin F,
N-(6-azido-D-galactosyl)-monomethylauristatin F,
N-(6-propargyl-D-galactosyl)-dolastatin 10,
N-(6-azido-D-galactosyl)-dolastatin 10,
N-(propargylgalactose)aminoepothilone, or
N-(2-deoxyglucosyl)aminoepothilone.

24. The cell binder-linker-payload molecule conjugate according to claim 4, wherein the cell binder comprises an antibody or a fragment thereof.

25. The linker-payload molecule conjugate according to claim 1, wherein Z is OH or a saccharide.

26. (canceled)

27. The linker-payload molecule conjugate according to claim 1, wherein the linker-payload molecule conjugate is
N-(2-deoxy-D-glucosyl)-monomethylauristatin F,
N-[6-O-(β-D-galacto-pyranosyl)-D-galactosyl]-monomethylauristatin F,
N-[4-O-(β-D-galactopyranosyl)-D-glucosyl]-monomethylauristatin F,
N-[2-acetamido-2-deoxy-4-O-(β-D-galactopyranosyl)-D-glucosyl)monomethylauristatin F,
N-{4-O-[4-O-(α-D-galactopyranosyl)-β-D-galactopyranosyl]-D-glucosyl}-monomethylauristatin F
N-(3-butynyl)-monomethylauristatin F,
N-(4-pentynyl)-monomethylauristatin F or
N-{6-O-[3-O-(α-N-acetylneuraminyl)-β-D-galactopyranosyl]-D-galactosyl}-monomethylauristatin F.

28. (canceled)

29. The cell binder-linker-payload molecule conjugate according to claim 4, wherein the cell binder-linker-payload molecule conjugate is selected from the group consisting of compounds of the following formulas:

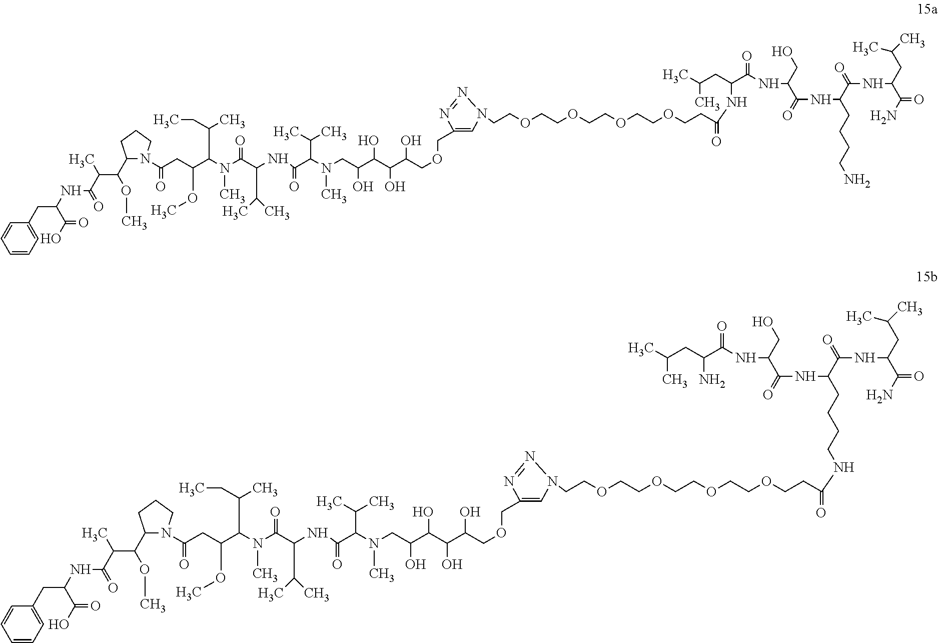 [SMILES: CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NC(CC(C)C)C(=O)NC(CO)C(=O)NC(CCCCN)C(=O)NC(CC(C)C)C(N)=O)nn1)C(C)C ; CCC(C)C(C(CC(=O)N1CCCC1C(OC)C(C)C(=O)NC(Cc1ccccc1)C(=O)O)OC)N(C)C(=O)C(NC(=O)C(C(C)C)N(C)CC(O)C(O)C(O)C(O)COCc1cn(CCOCCOCCOCCOCCC(=O)NCCCCC(NC(=O)C(CO)NC(=O)C(N)CC(C)C)C(=O)NC(CC(C)C)C(N)=O)nn1)C(C)C]

30. The cell binder-linker-payload molecule conjugate according to claim 24, wherein the antibody or a fragment thereof is directed against human vascular endothelial growth factor (VEGF), epidermal growth factor receptor 1 (EGFR), tumor necrosis factor alpha (TNF-α), CD20, CD22, HIV-1 envelope glycoprotein gp120, cancer-associated high-mannose type N-glycans, epidermal growth factor receptor 2 (HER2/neu), CD52, CD33, CD11a, glycoprotein IIb/IIIa, CD25, IgE, IL-2 receptor, or respiratory syncytial virus (RSV).

31. The cell binder-linker-payload molecule conjugate according to claim 24, wherein the antibody is cetuximab, trastuzumab, panitumumab, rituximab, bevacizumab, tositumomab, etanercept, adalimumab, alemtuzumab, gemtuzumab ozogamicin, efalizumab, rituximab, infliximab, abciximab, basiliximab, palivizumab, omalizumab, daclizumab, epratuzumab, lintuzumab, nimotuzumab, 2G12 or ibritumomab tiuxetan.

32. The cell binder-linker-payload molecule conjugate according to claim 4, wherein the cell binder-linker-payload molecule conjugate is a conjugate generated by azide-alkyne cycloaddition reaction between cetuximab-PEG₄-N₃ and N-(6-propargyl-D-galactosyl)-dolastatin 10, a conjugate generated by azide-alkyne cycloaddition reaction between omalizumab-PEG₄-N₃ and N-(6-propargyl-D-galactosyl)-dolastatin 10, a conjugate generated by azide-alkyne cycloaddition reaction between 2G12-PEG₄-N₃ and N-(6-propargyl-D-galactosyl)-dolastatin 10, or a conjugate generated by azide-alkyne cycloaddition reaction between trastuzumab-PEG₄-N₃ and N-(6-propargyl-D-galactosyl)-dolastatin 10.

33. A pharmaceutical composition comprising an effective amount of the linker-payload molecule conjugate according to claim 1, and a pharmaceutically acceptable carrier.

34. A method for modulating growth of a cell population, comprising the step of contacting the linker-payload conjugate according to claim 1 with the cell population.

35. (canceled)

36. (canceled)

37. (canceled)

38. (canceled)


SEQUENCE LISTING (33 sequences)

SEQ ID NO: 1 - protein, 1210 aa, Homo sapiens [sequence omitted]

SEQ ID NO: 2 - protein, 1255 aa, Homo sapiens [sequence omitted]

SEQ ID NO: 3 - protein, 449 aa, Artificial Sequence
  heavy chain, cetuximab, INN7906H, from IMGT
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Light chain, cetuximab, INN7906L, from IMGT
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, Artificial Sequence
  heavy chain, trastuzumab, 7637H, from IMGT
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 214 aa, Artificial Sequence
  light chain, trastuzumab, 7637L, from IMGT
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 451 aa, Artificial Sequence
  Heavy chain, 7609H, rituximab
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 213 aa, Artificial Sequence
  light chain, 7609L, rituximab
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 453 aa, Artificial Sequence
  8017H|bevacizumab|
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  8017L|bevacizumab|
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 467 aa, Artificial Sequence
  7783M|etanercept
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 121 aa, Artificial Sequence [sequence omitted]

SEQ ID NO: 13 - protein, 107 aa, Artificial Sequence [sequence omitted]

SEQ ID NO: 14 - protein, 451 aa, Artificial Sequence
  8005H|alemtuzumab
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, Artificial Sequence
  8005L|alemtuzumab
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 451 aa, Artificial Sequence
  8122H|efalizumab
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 214 aa, Artificial Sequence
  8122L|efalizumab
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 119 aa, Artificial Sequence
  7602H|infliximab
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVS

SEQ ID NO: 19 - protein, 107 aa, Artificial Sequence
  7602L|infliximab
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVK

SEQ ID NO: 20 - protein, 215 aa, Artificial Sequence
  3iu3A|basiliximab Fab|Chimeric||VH-CH1
QLQQSGTVLARPGASVKMSCKASGYSFTRYWMHWIKQRPGQGLEWIGAIYPGNSDTSYNQKFEGKAKLTAVTSASTAYMELSSLTHEDSAVYYCSRDYGYYFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEP

SEQ ID NO: 21 - protein, 210 aa, Artificial Sequence
  3iu3B|basiliximab Fab|Chimeric||L-KAPPA
QIVSTQSPAIMSASPGEKVTMTCSASSSRSYMQWYQQKPGTSPKRWIYDTSKLASGVPARFSGSGSGTSYSLTISSMEAEDAATYYCHQRSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 22 - protein, 451 aa, Artificial Sequence
  8039H|omalizumab|Humanized
EVQLVESGGGLVQPGGSLRLSCAVSGYSITSGYSWNWIRQAPGKGLEWVASITYDGSTNYNPSVKGRITISRDDSKNTFYLQMNSLRAEDTAVYYCARGSHYFGHWHFAVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 218 aa, Artificial Sequence
  8039L|omalizumab
DIQLTQSPSSLSASVGDRVTITCRASQSVDYDGDSYMNWYQQKPGKAPKLLIYAASYLESGVPSRFSGSGSGTDFTLTISSLQPEDFATYYCQQSHEDPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 446 aa, Artificial Sequence
  7164H|daclizumab
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 213 aa, Artificial Sequence
  7164L|daclizumab
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 453 aa, Artificial Sequence
  nimotuzumab_HC
QVQLQQSGAEVKKPGSSVKVSCKASGYTFTNYYIYWVRQAPGQGLEWIGGINPTSGGSNFNEKFKTRVTITVDESTNTAYMELSSLRSEDTAFYFCARQGLWFDSDGRGFDFWGQGSTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 219 aa, Artificial Sequence
  nimotuzumab_LC
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITREVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 446 aa, Artificial Sequence
  epratuzumab_HC
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 219 aa, Artificial Sequence
  epratuzumab_LC
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 446 aa, Artificial Sequence
  lintuzumab_HC
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 217 aa, Artificial Sequence
  lintuzumab_LC
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 214 aa, Artificial Sequence
  2G12_LC
DVVMTQSPSTLSASVGDTITITCRASQSIETWLAWYQQKPGKAPKLLIYKASTLKTGVPSRFSGSGSGTEFTLTISGLQFDDFATYHCQHYAGYSATFGQGTRVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 453 aa, Artificial Sequence
  2G12_HC
EVQLVESGGGLVKAGGSLILSCGVSNFRISAHTMNWVRRVPGGGLEWVASISTSSTYRDYADAVKGRFTVSRDDLEDFVYLQMHKMRVEDTAIYYCARKGSDRLSDNDPFDAWGPGTVVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK